(19) United States
(12) Patent Application Publication
(10) Pub. No.: US 2023/0010999 A1
(43) Pub. Date: Jan. 12, 2023

(54) ABSORBENT ARTICLE

(71) Applicant: Kimberly-Clark Worldwide, Inc., Neenah, WI (US)

(72) Inventors: Jason K. Sieck, Neenah, WI (US); Andrew J. Nelson, Neenah, WI (US); Gregory A. Zander, Fremont, WI (US)

(21) Appl. No.: 17/782,224

(22) PCT Filed: Jan. 5, 2021
(86) PCT No.: PCT/US2021/012149

§ 371 Date: Jun. 3, 2022

Related U.S. Application Data
(60) Provisional application No. 62/957,490, filed on Jan. 6, 2020.

(51) Int. Cl. A61F 13/53; A61F 13/49; A61F 13/496

(52) U.S. Cl. CPC A61F 13/53; A61F 13/49011; A61F 13/4902; A61F 13/496; A61F 2013/49025

(57) ABSTRACT

An absorbent article, such as, a garment-like absorbent article, can have a first waist region, a second waist region, and a crotch region extending between and interconnecting the first waist region and the second waist region. Each of the first waist region and the second waist region can have an elastic material. The absorbent article can have an absorbent core wherein a portion of the absorbent core can be located in the crotch region, a portion of the absorbent core can be located in a portion of the first waist region, and a portion of the absorbent core can be located in a portion of the second waist region. Within each of the first waist region and the second waist region, an overlapping region can exist where the absorbent core is in an overlapping configuration with the elastic material within the first waist region and the second waist region.

### BACKGROUND OF THE DISCLOSURE

Child care, feminine care, and adult hygiene-related absorbent personal care articles are often used to protect a wearer's outer garments from soiling, and to collect and retain body exudates such as menses, blood, feces, and urine. Such articles are often presented in disposable garment-like product formats (as opposed to inserts, pads, or liners) and are worn as undergarments in the place of traditional underwear. They are most commonly placed on a wearer by being pulled up about a wearer's legs towards the wearer's lower abdomen and placed adjacent a wearer's crotch region during use.

Today, many wearers of absorbent garment-like articles include adults who experience various forms of incontinence. Primary desired attributes of such garments include the garment retaining body exudate, minimal or no leakage of body exudate, close-to-body fit of the garment, and that it resembles traditional woven underwear. Consumers are interested in such attributes as there is a desire to enhance the overall personal experience of using such products while reducing incontinence-related stigma. Consumers want a garment that will meet their needs without signaling to others that they are wearing such absorbent garment-like articles. Absorbent article stigmas are aggravated by product designs which can feel bulky when in use, are ill-fitting and uncomfortable for the wearer to wear, do not fit close to the body and, therefore, may gap away from the wearer's body producing an outline that can be seen through a wearer's clothing, may be manufactured from materials that can create relatively high levels of noise during use due to the specific product construction materials, and by an overall artificial visual appearance of such products when viewed by the wearer and also by third parties.

In order to improve the fit of garment-like articles, many garment-like articles are formed by positioning an absorbent assembly between or otherwise bonded to at least one stretchable or elastomeric outer layer of the garment-like article. Garments with active elastic materials positioned over and around the absorbent material can result in the absorbent material bunching up. Such bunching of the absorbent material can create fit and discretion problems. From a fit standpoint, bunched up absorbent material is less likely to lie snugly against the body, potentially increasing the incidence of leakage. From a discretion standpoint, excessive bunching tends to make the product more bulky and therefore more visible under clothing. This circumstance is particularly problematic for incontinence articles, such as enuresis pants and adult pull-on style disposable absorbent underwear, as the wearers of such products generally are embarrassed about their condition and wish to employ protection which is as discreet as possible.

There is a need for an absorbent article having an improved fit about the lower torso of the wearer. There is a need for an absorbent article having elasticized waist regions that are less likely to cause undesirable gathering and bunching of the absorbent material.

### SUMMARY OF THE DISCLOSURE

In various embodiments, an absorbent article can have a longitudinal direction and a transverse direction; a longitudinal axis and a transverse axis; a first waist region comprising a first waist edge, a first longitudinal direction side edge, a second longitudinal direction side edge transversely opposed to the first longitudinal direction side edge, a first elastomeric panel comprising a first elastic material positioned between a first nonwoven material and a second nonwoven material wherein the first elastic material is elastomeric in the transverse direction; a second waist region comprising a second waist edge, a third longitudinal direction side edge, a fourth longitudinal direction side edge transversely opposed to the third longitudinal direction side edge, a second elastomeric panel comprising a second elastic material positioned between a third nonwoven material and a fourth nonwoven material, wherein the second elastic material is elastomeric in the transverse direction; a first side seam formed by bonding the first longitudinal direction side edge of the first waist region to the third longitudinal direction side edge of the second waist region and a second side seam formed by bonding the second longitudinal direction side edge of the first waist region to the fourth longitudinal direction side edge of the second waist region; a crotch region located between the first waist region and the second waist region and interconnecting the first waist region and the second waist region; an article length measured from the first waist edge to the second waist edge; and an absorbent assembly can have an absorbent core comprising a first longitudinal direction side edge and a second longitudinal direction side edge transversely opposed to the first longitudinal direction side edge, a topsheet layer facing surface and a backsheet layer facing surface, wherein a first portion of the absorbent core is located in the crotch region, a second portion of the absorbent core is located in a portion of the first waist region and in an overlapping configuration with a portion of the first elastic material and comprises a first maximum transverse direction width, and a third portion of the absorbent core is located in a portion of the second waist region and in an overlapping configuration with a portion of the second elastic material and comprises a second maximum transverse direction width; a core wrap comprising a first side edge spaced apart from the first longitudinal direction side edge of the absorbent core by a distance of at least 3.5 mm in at least one of the first waist region or the second waist region when the absorbent article is in a transverse direction fully extended configuration, and a second side edge spaced apart from the second longitudinal direction side edge of the absorbent core by a distance of at least 3.5 mm in at least one of the first waist region or the second waist region when the absorbent article is in a transverse direction fully extended configuration; and wherein a portion of the core wrap is in a face-to-face relationship with the backsheet layer facing surface of the absorbent core.

In various embodiments, the first elastic material and the second elastic material is a plurality of elastomeric strands. In various embodiments, the first elastic material and the second elastic material is a polymeric film sheet.

In various embodiments, the first maximum transverse direction width of the absorbent core is greater than the second maximum transverse direction width of the absorbent core. In various embodiments, the first maximum transverse direction width of the absorbent core is the same as the second maximum transverse direction width of the absorbent core. In various embodiments, the first maximum transverse direction width of the absorbent core is less than the second maximum transverse direction width of the absorbent core.

In various embodiments, in the second waist region, the first side edge of the core wrap is spaced apart from the first longitudinal direction side edge of the absorbent core at the second maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the second maximum transverse direction width of the absorbent core by a distance of at least 3.5 mm when the absorbent article is in a transverse direction fully extended configuration. In various of such embodiments, in the first waist region, the first side edge of the core wrap is spaced apart from the first longitudinal direction side edge of the absorbent core at the first maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the first maximum transverse direction width of the absorbent core by a distance of at least 3.5 mm when the absorbent article is in a transverse direction fully extended configuration.

In various embodiments, in each of the first waist region and the second waist region, the first side edge of the core wrap is spaced apart from the first longitudinal direction side edge of the absorbent core at each of the first maximum transverse direction width and the second maximum transverse direction width by a distance of at least 3.5 mm when the absorbent article is in a transverse direction fully extended configuration and the second side edge of the core wrap is spaced apart from the second longitudinal direction side edge of the absorbent core at each of the first maximum transverse direction width and the second maximum transverse direction width by a distance of at least 3.5 mm when the absorbent article is in a transverse direction fully extended configuration.

In various embodiments, in the first waist region, the first side edge of the core wrap is spaced apart from the first longitudinal direction side edge of the absorbent core at the first maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the first maximum transverse direction width of the absorbent core by a distance of at least 3.5 mm when the absorbent article is in a transverse direction fully extended configuration. In various of such embodiments, in the second waist region, the first side edge of the core wrap is spaced apart from the first longitudinal direction side edge of the absorbent core at the second maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the second maximum transverse direction width of the absorbent core by a distance of at least 3.5 mm when the absorbent article is in a transverse direction fully extended configuration.

In various embodiments, the absorbent article can further have an absorbent article narrowest width in the transverse direction positioned in the crotch region and between the transverse axis and the first waist edge. In various embodiments, the absorbent article can further have a first waist region width in the transverse direction from the first longitudinal direction side edge to the second longitudinal direction side edge wherein the absorbent article narrowest width is less than 25% of the first waist region width. In various embodiments, the absorbent article can further have a first article sub-length measured from the first waist edge to the absorbent article narrowest width and a second article sub-length measured from the second waist edge to the absorbent article narrowest width wherein the first article sub-length is less than the second article sub-length. In various embodiments, the first article sub-length is less than 45% of the article length and the second article sub-length is greater than 55% of the article length.

In various embodiments, the absorbent core can further have an absorbent core midpoint which is positioned between the absorbent article narrowest width and the transverse axis. In various embodiments, the absorbent core midpoint is offset in the longitudinal direction from the absorbent article narrowest width by a distance from 1% to 5% of the article length. In various embodiments, the absorbent core midpoint is offset in the longitudinal direction from the transverse axis of the absorbent article by a distance from 6% to 15% of the article length.

### BRIEF DESCRIPTION OF DRAWINGS

FIG. 1 is an illustration of a front view of an embodiment of an absorbent article in a pull-on, pant-like configuration.

FIG. 2 is an illustration of a back view of the absorbent article of FIG. 1.

FIG. 3 is an illustration of a plan view of an embodiment of the absorbent article of FIG. 1 in a longitudinally and transversely stretched and laid-flat configuration prior to the joining of the first and second waist regions with the surface of the absorbent article that faces the wearer when the absorbent article is worn facing the viewer.

FIG. 4 is an illustration of a cross-sectional view of an embodiment of the absorbent article of FIG. 3 taken along line 4-4.

FIGS. 5A and 5B are photographs of portions of an absorbent article in a relaxed configuration.

FIG. 6A is a schematic illustration of a portion of an absorbent article in a transverse direction stretched configuration.

FIG. 6B is a schematic illustration of the portion of the absorbent article of FIG. 6A in a relaxed configuration.

FIG. 7 is a photograph of a portion of an absorbent article in a relaxed configuration.

FIG. 8A is an illustration of an embodiment of an absorbent core and a core wrap.

FIGS. 8B and 8C are cross-sectional views of the absorbent core and core wrap of FIG. 8A taken along lines 8B-8B and 8C-8C, respectively.

FIG. 9A is an illustration of an embodiment of an absorbent core and a core wrap.

FIGS. 9B and 9C are cross-sectional views of the absorbent core and core wrap of FIG. 9A taken along lines 9B-9B and 9C-9C, respectively.

FIG. 10A is an illustration of an embodiment of an absorbent core and a core wrap.

FIGS. 10B and 10C are cross-sectional views of the absorbent core and core wrap of FIG. 10A taken along lines 10B-10B and 10C-10C, respectively.

FIG. 11A is an illustration of an embodiment of an absorbent core and a core wrap.

FIGS. 11B and 11C are cross-sectional views of the absorbent core and core wrap of FIG. 11A taken along lines 11B-11B and 11C-11C, respectively.

FIG. 12A is an illustration of an embodiment of an absorbent core and a core wrap.

FIGS. 12B and 12C are cross-sectional views of the absorbent core and core wrap of FIG. 12A taken along lines 12B-12B and 12C-12C, respectively.

FIG. 13A is an illustration of an embodiment of an absorbent core and a core wrap.

FIGS. 13B and 13C are cross-sectional views of the absorbent core and core wrap of FIG. 13A taken along lines 13B-13B and 13C-13C, respectively.

FIG. 14 is an illustration of a cross-sectional view of an alternate embodiment of the absorbent article of FIG. 3 taken along line 14-14.

FIG. 15 is an illustration of a plan view of an alternate embodiment of an absorbent article in a longitudinally and transversely stretched and laid-flat configuration prior to the joining of the first and second waist regions with the surface of the absorbent article that faces the wearer when the absorbent article is worn facing the viewer.

FIG. 16 is an illustration of a cross-sectional view of an embodiment of the absorbent article of FIG. 15 taken along line 16-16.

Repeat use of reference characters in the present specification and drawings is intended to represent the same or analogous features or elements of the disclosure.

### DETAILED DESCRIPTION OF THE DISCLOSURES

The present disclosure is directed towards an absorbent article such as a garment-like absorbent article. The absorbent article has a first waist region, a second waist region, and a crotch region extending between and interconnecting the first waist region and the second waist region. Each of the first waist region and the second waist region can have an elastic material. The absorbent article can have an absorbent core wherein a portion of the absorbent core can be located in the crotch region, a portion of the absorbent core can be located in a portion of the first waist region, and a portion of the absorbent core can be located in a portion of the second waist region. Within each of the first waist region and the second waist region, an overlapping region can exist where the absorbent core is in an overlapping configuration with the elastic material within the first waist region and the second waist region.

As used herein, the term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, training pants, youth pants, swim pants, enuresis garments, menstrual pants, and adult incontinence garments, and the like without departing from the scope of the present disclosure.

As used herein, the term “airlaid” refers herein to a web manufactured by an airlaying process In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form on fiber. Conjugate fibers are also sometimes referred to as bicomponent or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al., each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Buten, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10, or 20 gsm to about 120, 125, or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent,” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in par on iconicity (a factor of the ionic concentration of the aqueous solution), and the SAP functional polar groups that have an affinity for water. SAP are typically made from the polymerization of acrylic acid blended with sodium hydroxide I the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating or another material or fiber.

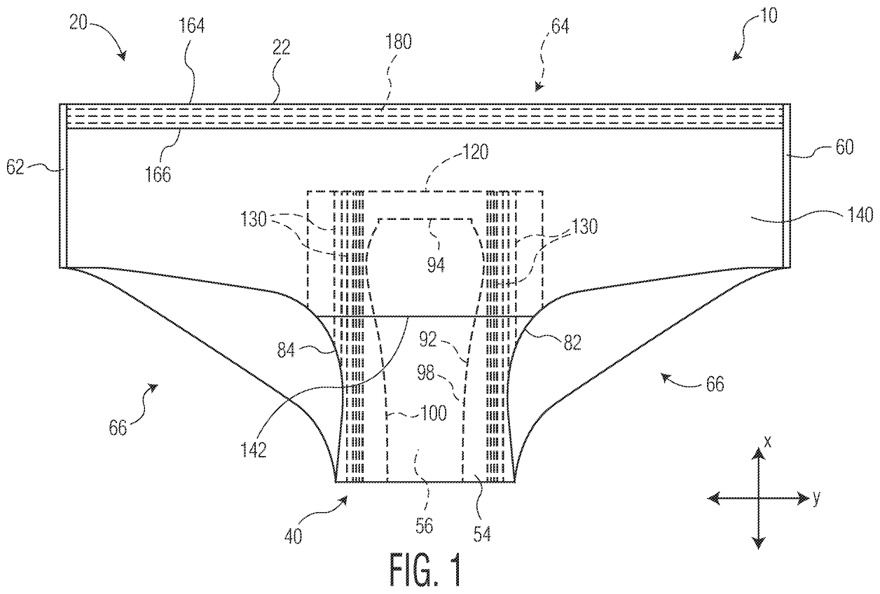

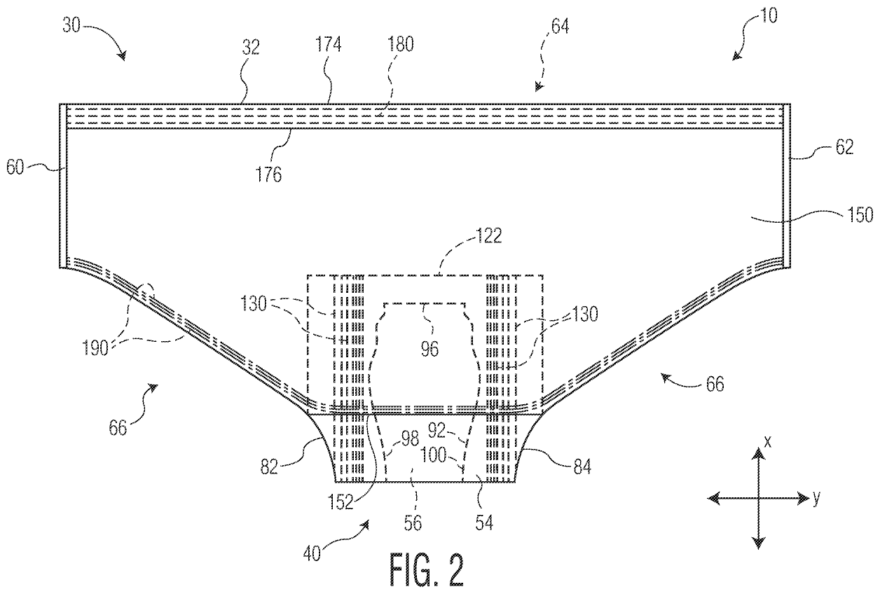

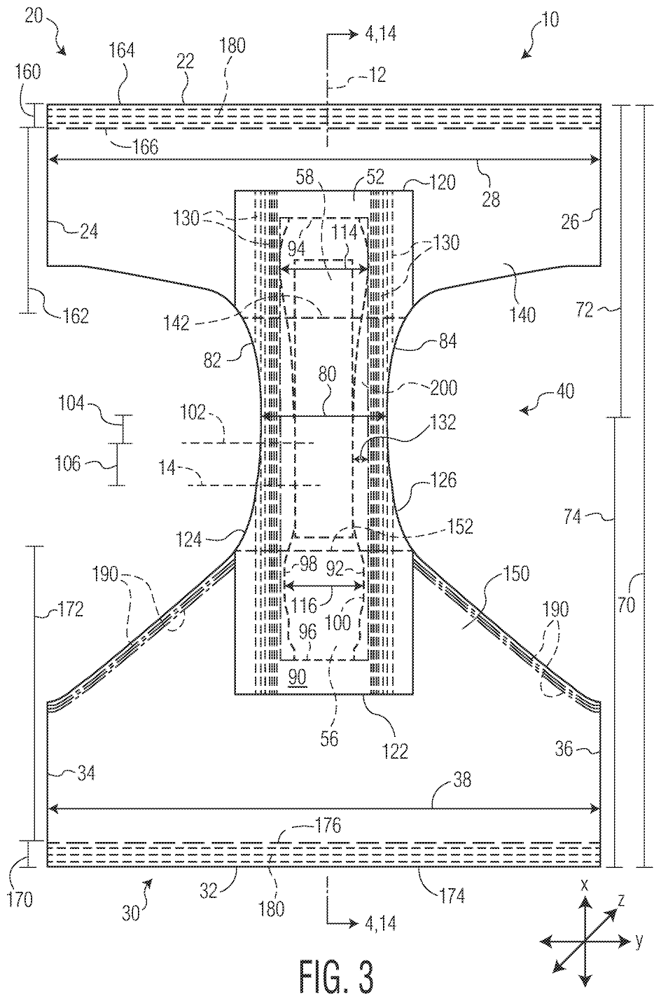

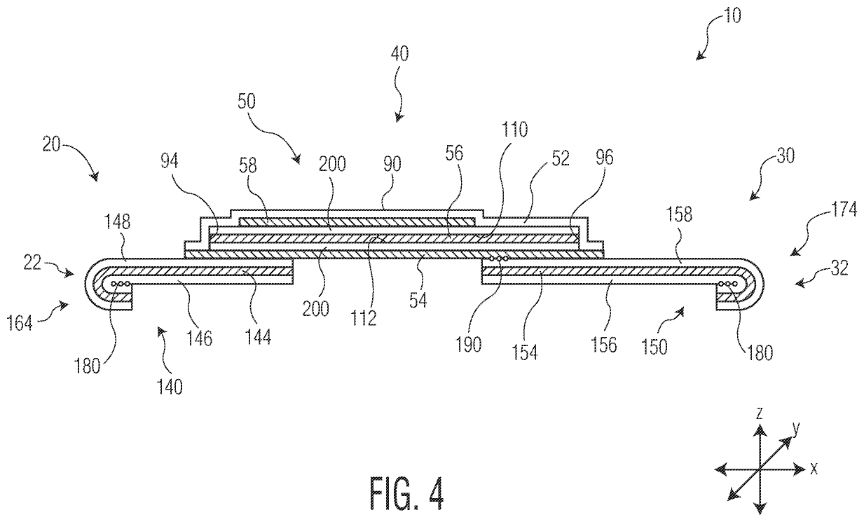

Referring to FIGS. 1-4, an illustration of an exemplary embodiment of an absorbent article 10 is illustrated. FIG. 1 provides an illustration of an embodiment of a front view of the absorbent article 10 in a pull-on, pant-like configuration, FIG. 2 provides an illustration of a back view of the absorbent article 10 of FIG. 1 in a pull-on, pant-like configuration, FIG. 3 provides an illustration of a plan view of an embodiment of the absorbent article 10 of FIG. 1 in a longitudinally and transversely stretched and laid-flat configuration prior to the joining of the first waist region 20 and second waist region 30 with the surface of the absorbent article 10 that faces the wearer when the absorbent article 10 is worn facing the viewer, and FIG. 4 provides an illustration of a cross-sectional view of an embodiment of the absorbent article 10 of FIG. 3 taken along line 4-4. Although for illustrative purposes certain features of the present disclosure can be described and illustrated with respect to an adult incontinence garment, the various aspects and embodiments of the present disclosure are also suitable for use with diapers, youth pants, swim pants, training pants, enuresis garments, menstrual pants, and the like.

The absorbent article 10 has a longitudinal direction (X), a transverse direction (Y), and a depth direction (Z). The absorbent article 10 can have a longitudinal axis 12 and a transverse axis 14. The absorbent article 10 is intended to be worn about the lower torso of a human and can have a first waist region 20, a second waist region 30, and a crotch region 40 extending between and interconnecting the first waist region 20 and the second waist region 30. The first waist region 20 and the second waist region 30 are those regions of the absorbent article 10 that are fitted circumferentially around at least the anterior and posterior regions of the lower torso of the wearer of the absorbent article 10 including, for example, the wearer's abdomen, lower back, buttock, and hips. The crotch region 40 of the absorbent article 10 is that region of the absorbent article 10 that will be positioned between the wearer's legs when the absorbent article 10 is fitted onto the wearer.

The first waist region 20 has a first waist edge 22, a first longitudinal direction side edge 24, and a second longitudinal direction side edge 26 transversely opposed to the first longitudinal direction side edge 24. The second waist region 30 has a second waist edge 32, a first longitudinal direction side edge 34, and a second longitudinal direction side edge 36 transversely opposed to the first longitudinal direction side edge 34. To place the absorbent article 10 into a suitable configuration for wearing about the lower torso of the wearer, the first longitudinal direction side edge 24 of the first waist region 20 can be bonded to the first longitudinal direction side edge 34 of the second waist region 30 to form a first side seam 60 and the second longitudinal direction side edge 26 of the first waist region 20 can be bonded to the second longitudinal direction side edge 36 of the second waist region 30 to form a second side seam 62. Forming the side seams, 60 and 62, can create a wearable absorbent article 10 having a waist opening 64 and a pair of leg openings 66.

The first waist region 20 can have a first waist region width 28 measured in the transverse direction (Y) between the first and second longitudinal direction side edges, 24 and 26, of the first waist region 20. The first waist region width 28 is measured with the absorbent article 10 fully extended in the transverse direction (Y) such as illustrated in FIG. 3 when the absorbent article 10 is in a longitudinally and transversely stretched and laid-flat configuration prior to the joining of the first waist region 20 and second waist region 30 to form the side seams, 60 and 62. As used herein, the term “fully extended” describes the condition wherein the absorbent article 10 is extended in a given direction to the point where any further extension in said direction would result in one or more material failures (e.g., rupture or permanent deformation). In embodiments wherein the first longitudinal direction side edge 24 and the second longitudinal direction side edge 26 are not parallel with the longitudinal direction (X) (not illustrated), the first waist region width 28 is the maximum width measured parallel with the transverse direction (Y) from any point on the first longitudinal direction side edge 24 to any point on the second longitudinal direction side edge 26. In various embodiments, the first waist region width 28 may be from 600 or 625 mm to 850 or 900 mm.

The second waist region 30 can have a second waist region width 38 measured in the transverse direction (Y) between the first and second longitudinal direction side edges, 34 and 36, of the second waist region 30. The second waist region width 38 is measured with the absorbent article 10 fully extended in the transverse direction (Y) such as illustrated in FIG. 3 when the absorbent article 10 is in a longitudinally and transversely stretched and laid-flat configuration prior to the joining of the first waist region 20 and second waist region 30 to form the side seams, 60 and 62. In embodiments wherein the first longitudinal direction side edge 34 and the second longitudinal direction side edge 36 are not parallel with the longitudinal direction (X) (not illustrated), the second waist region width 38 is the maximum width measured parallel with the transverse direction (Y) from any point on the first longitudinal direction side edge 34 to any point on the second longitudinal direction side edge 36. In various embodiments, the second waist region width 38 may be from 600 or 625 mm to 850 or 900 mm.

The absorbent article 10 has an article length 70 as measured in the longitudinal direction (X) from the first waist edge 22 to the second waist edge 32 as illustrated in FIG. 3. The article length 70 is measured with the absorbent article 10 fully extended in the longitudinal direction (X) such as illustrated in FIG. 3 when the absorbent article 10 is in a longitudinally and transversely stretched and laid-flat configuration prior to the joining of the first waist region 20 and second waist region 30 to form the side seams, 60 and 62. In various embodiments, the article length 70 may be at least 600, 620, 640, 660, 680, 700, 720, 740, 760, 780, 800, 820, 840, 860, 880, or 900 mm.

The crotch region 40 is disposed in the longitudinal direction (X) between and interconnecting the first waist region 20 and the second waist region 30. The absorbent article has an absorbent article narrowest width 80 located within the crotch region 40 of the absorbent article 10. The absorbent article narrowest width 80 is measured in the transverse direction (Y) as the narrowest dimension between a first leg edge 82 and a second leg edge 84 such as illustrated in FIG. 3. The absorbent article narrowest width 80 is measured with the absorbent article 10 fully extended in a longitudinally and transversely stretched and laid-flat configuration prior to the joining of the first waist region 20 and second waist region 30 to form the side seams, 60 and 62. In various embodiments, the absorbent article narrowest width 80 is less than 25% or 20% of the first waist region width 28. For example, in various embodiments, the absorbent article narrowest width 80 may be about 150 mm and the first waist region width 28 may be about 630 mm or 740 mm. In such embodiments, the absorbent article narrowest width 80 may be about 24% or 20% of the first waist region width 28, respectively. As another example, in various embodiments, the absorbent article narrowest width 80 may be about 150 mm and the first waist region width 80 may be about 750 mm or 880 mm. In such embodiments, the absorbent article narrowest width 80 may be about 20% or 17% of the first waist region width 28, respectively. The lower the percentage of the absorbent article narrowest width 80, relative to the first waist region width 28, the more shaped the absorbent article 10 is within the crotch region 40. In other words, the higher the percentage (up to 100%) the more rectangular the absorbent article 10 is within the crotch region 40. An absorbent article 10 having a more rectangular shape within the crotch region 40 may provide too much bulk of an absorbent article 10 between the wearer's legs which fails to follow the contours of the wearer's legs. This can result in the absorbent article 10 bunching up between the wearer's legs, protruding away from the body of the wearer, and not fitting close to the body of the wearer at the location where body exudate exits the body of the wearer. Providing a non-rectangular shape to the absorbent article 10 within the crotch region 40 of the absorbent article 10 can remove bulk from between the wearer's legs and allow the absorbent article 10 to better fit against and between the contours of the wearer's legs. This can allow for improved conformance of the absorbent article 10 to the body of the wearer.

In various embodiments, the absorbent article narrowest width 80 is positioned between the first waist edge 22 and the transverse axis 14 of the absorbent article 10. In such embodiments, the absorbent article narrowest width 80 is not in an overlapping alignment with the transverse axis 14 of the absorbent article 10. The absorbent article narrowest width 80 can apportion the article length 70 into a first article sub-length 72 and a second article sub-length 74. The first article sub-length 72 can be measured in the longitudinal direction (X) from the first waist edge 22 to the absorbent article narrowest width 80 when the absorbent article 10 is in a longitudinally and transversely stretched and laid-flat configuration prior to the joining of the first waist region 20 and second waist region 30 to form the side seams, 60 and 62. The second article sub-length 74 can be measured in the longitudinal direction (X) from the second waist edge 32 to the absorbent article narrowest width 80 when the absorbent article 10 is in a longitudinally and transversely stretched and laid-flat configuration prior to the joining of the first waist region 20 and second waist region 30 to form the side seams, 60 and 62. In various embodiments, the first article sub-length 72 is less than the second article sub-length 74. In various embodiments, the first article sub-length 72 can be less than 45% or 40% of the total article length 70. In various embodiments, the second article sub-length 74 can be greater than 55% or 60% of the total article length 70. Wearers of absorbent articles 10 have body shapes in a variety of shapes, sizes, and curvature, and are generally not symmetrical. Placing a symmetrical about the transverse axis 14 absorbent article 10 on a body which is not symmetrical can result in a reduction in proper fit of the absorbent article 10 on the body of the wearer. Positioning the absorbent article narrowest width 80 closer to the first waist edge 22, and not in alignment with the transverse axis 14, can provide for a non-symmetrical about the transverse axis 14 absorbent article 10. As a result, when the absorbent article narrowest width 80 is positioned between the legs of the wearer a greater proportion of the absorbent article 10 is positioned on the posterior side of the wearer's body providing for better coverage of the buttocks of the wearer of the absorbent article 10.

The absorbent article 10 can also include an absorbent assembly 50. The absorbent assembly 50 can extend in the longitudinal direction (X) of the absorbent article 10 from the first waist region 20, through the crotch region 40, and to the second waist region 30. In various embodiments, the absorbent assembly 50 can have at least a topsheet layer 52, a backsheet layer 54, and an absorbent core 56 positioned between the topsheet layer 52 and the backsheet layer 54. In various embodiments, the absorbent assembly 50 can have at least a topsheet layer 52, a backsheet layer 54, an absorbent core 56 positioned between the topsheet layer 52 and the backsheet layer 54, and a surge layer 58 positioned between the absorbent core 56 and the topsheet layer 52. In various embodiments, the absorbent core 56 can be at least partially enveloped by a core wrap 200. The topsheet layer 52 can be bonded to the backsheet layer 54 beyond the outermost edge of the absorbent core 56 to form a perimeter seal for the absorbent assembly 50. The perimeter seal can contain the body exudates within the absorbent assembly 50 of the absorbent article 10.

The topsheet layer 52 defines a body facing surface 90 of the absorbent assembly 50 that may directly contact the body of the wearer and is liquid permeable to receive body exudates. The topsheet layer 52 is desirably provided for comfort and functions to direct body exudates away from the body of the wearer, through its own structure, and towards the absorbent core 56. The topsheet layer 52 desirably retains little to no liquid in its structure, so that it provides a relatively comfortable and non-irritating surface next to the skin of the wearer of the absorbent article 10.

The topsheet layer 52 can be a single layer of material, or alternatively, can be multiple layers that have been laminated together. The topsheet layer 52 can be constructed of any material such as one or more woven sheets, one or more fibrous nonwoven sheets, one or more aperture film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 52.

In various embodiments the topsheet layer 52 can be constructed from various nonwoven webs such as meltblown webs, spunbond webs, hydroentangled spunlace webs, or through air bonded carded webs. Examples of suitable topsheet layer 52 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 52 can be a bonded carded web made of polypropylene and polyethylene such as that obtainable from Sandler Corp., Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 52, each of which is hereby incorporated by reference thereto in its entirety. Additional topsheet layer 52 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 52 may contain a plurality of apertures formed therethrough to permit body exudates to pass more readily into the absorbent core 56. The apertures may be randomly or uniformly arranged throughout the topsheet layer 52. The size, shape, diameter, and number of apertures may be varied to suit an absorbent article's 10 particular needs.

In various embodiments, the tospheet layer 52 can have a basis weight ranging from about 5, 10, 15, 20, or 25 gsm to about 50, 100, 120, 125, or 150 gsm. For example, in an embodiment, a topsheet layer 52 can be constructed from a through air bonded carded web having a basis weight ranging from about 15 gsm to about 100 gsm. In another example, a topsheet layer 52 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics, and others.

In various embodiments, the topsheet layer 52 can be at least partially hydrophilic. In various embodiments, a portion of the topsheet layer 52 can be hydrophilic and a portion of the topsheet layer 52 can be hydrophobic. In various embodiments, the portions of the topsheet layer 52 which can be hydrophobic can be either an inherently hydrophobic material or can be a material treated with a hydrophobic coating.

In various embodiments, the topsheet layer 52 can be a multicomponent topsheet layer 52 such as by having two or more different nonwoven or film materials, with the different materials placed in separate locations in the transverse direction (Y) of the absorbent assembly 50. For example, the topsheet layer 52 can be a two layer or multicomponent material having a central portion positioned along and straddling a longitudinal axis 12 of an absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion of a topsheet layer 52 can be positioned symmetrically about the absorbent article 10 longitudinal axis 12. Such central longitudinally directed central portion can be a through air bonded carded web (“TABCW”) having a basis weight between about 15 and about 100 gsm. Previously described nonwoven, woven, and aperture film topsheet layer materials may also be used as the central portion of a topsheet layer 52. In various embodiments, the central portion can be constructed from a TABCW material having a basis weight from about 20 gsm to about 50 gsm such as is available from Xiamen Yanjan Industry, Beijing, DaYuan Nonwoven Fabrics, and others. Alternatively, aperture films, such as those available from such film suppliers as Texol, Italy and Tredegar, U.S.A. may be utilized. Different nonwoven, woven, or film sheet materials may be utilized as the side portions of the topsheet layer 52. The selection of such topsheet layer 52 materials can vary based upon the overall desired attributes of the topsheet layer 52. For example, it may be desired to have a hydrophilic material in the central portion and hydrophobic-barrier type materials in the side portions to prevent leakage and increase a sense of dryness in the area of the side portions. Such side portions can be adhesively, thermally, ultrasonically, or otherwise bonded to the central portion along or adjacent the longitudinally directed side edges of the central portion. Traditional absorbent article construction adhesive may be used to bond the side portions to the central portion. Either of the central portion and/or the side portions may be treated with surfactants and/or skin-health benefit agents, as are well known in the art.

Such longitudinally directed side portions can be of a single or multi-layered construction. In various embodiments, the side portions can be adhesively or otherwise bonded laminates. In various embodiments, the side portions can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer 52 design, it may include opacifying agents, such as film pigments, that can help the film in masking stains along the absorbent article 10 side edges, thereby serving as a masking element. In such a fashion, the film layer can serve to limit visualization of a fluid insult stain along the absorbent assembly 50 side edges when viewed from above the topsheet layer 52. The film layer may also serve as a barrier layer to prevent rewet of the topsheet layer 52 as well as to prevent the flow of fluid off the side edges of the absorbent assembly 50. In various embodiments, the side portions can be laminates such as a spunbond-meltblown-meltblown-spunbond layer (“SMMS”) laminate, spunbond-film laminate, or alternatively, other nonwoven laminate combinations.

The backsheet layer 54 of the absorbent assembly 50 is generally liquid impermeable and is the portion of the absorbent assembly 50 which faces the garments of the wearer. The backsheet layer 54 can permit the passage of air or vapor out of the absorbent article 10 while still blocking the passage of liquids. Any liquid impermeable material may generally be utilized to form the backsheet layer 54. The backsheet layer 54 can be composed of a single layer or multiple layers, and these one or more layers can themselves comprise similar or different materials. Suitable material that may be utilized can be a microporous polymeric film, such as a polyolefin film or polyethylene or polypropylene, nonwovens, and nonwoven laminates, and film/nonwoven laminates. The particular structure and composition of the backsheet layer 54 can be selected from various known films and/or fabrics with the particular material being selected as appropriate to provide the desired level of liquid barrier, strength, abrasion resistance, tactile properties, aesthetics, and so forth. In various embodiments, a polyethylene film can be utilized that can have a thickness in the range of from about 0.2 or 0.5 mils to about 3.0 or 5.0 mils. An example of a backsheet layer 54 can be a polyethylene film such as that obtainable from Pliant Corp., Schaumburg, Ill., USA. Another example can include calcium carbonate-filled polypropylene film. In still another embodiment, the backsheet layer 54 can be a hydrophobic nonwoven material with water barrier properties such as a nonwoven laminate, an example of which can be a spunbond, meltblown, meltblown, spunbons, four-layered laminate.

In various embodiments, the backsheet layer 54 can be a two layer construction, including an outer layer material and an inner layer material which can be bonded together. The outer layer can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer can be a 20 gsm spunbond polypropylene non-woven web. The inner layer can be either vapor permeable (i.e., “breathable”) or vapor impermeable. The inner layer may be manufactured from a thin plastic film, although other liquid impermeable materials may also be used. The inner layer can inhibit liquid body exudates from leaking out of the absorbent assembly 50 and wetting articles, such as bed sheets and clothing, as well as the wearer and caregiver. An example of a material for an inner layer can be a printed 19 gsm Berry Plastics XP-8695H film or equivalent commercially available from Berry Plastics Corporation, Evansville, Ind., U.S.A.

The backsheet layer 54 can, therefore, be of a single or multiple layer construction, such as of multiple film layers or laminates of film and nonwoven fibrous layers. Suitable backsheet layers 206 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

An absorbent core 56 can be positioned between the topsheet layer 52 and the backsheet layer 54 of the absorbent article 10. In various embodiments, the absorbent core 56 can extend in the longitudinal direction (X) of the absorbent assembly 50. The absorbent core 56 can have a first portion located in the crotch region 40 of the absorbent article 10. In various embodiments, the absorbent core 56 can have a second portion located in a portion of at least one of the first waist region 20 or the second waist region 30. In various embodiments, the absorbent core 56 can have a first portion located within the crotch region 40 and a second portion located in a portion of the first waist region 20. In various embodiments, the absorbent core 56 can have a first portion located within the crotch region 40 and a second portion located in a portion of the second waist region 30. In various embodiments, an absorbent core 56 can have a first portion located within the crotch region 40, a second portion located in a portion of the first waist region 20, and a third portion located in a portion of the second waist region 30.

The absorbent core 56 can generally be any single layer structure or combination of layer components, which can demonstrate some level of compressibility, conformability, be non-irritating to the wearer's skin, and capable of absorbing and retaining liquids and other body exudates. In various embodiments, the absorbent core 56 can be formed from a variety of different materials and can contain any number of desired layers. For example, the absorbent core 56 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of wood pulp fluff can be identified with the trade designation NB416, available from Weyerhaeuser Corp., and is a bleached, highly absorbent wood pulp containing primarily soft wood fibers.

In various embodiments, if desired, the absorbent core 56 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 56 in any amount as desired.

Regardless of the combination of absorbent materials used in the absorbent core 56, the absorbent materials can be formed into a web structure by employing various conventional methods and techniques. For example, the absorbent web can be formed by techniques such as, but not limited to, a dry-forming technique, an air forming technique, a wet forming technique, a foam forming technique, or the like, as well as combinations thereof. A coform nonwoven material can also be employed. Methods and apparatus for carrying out such techniques are well known in the art.

The absorbent core 56 can have a perimeter edge 92 formed by a first transverse direction end edge 94, a second transverse direction end edge 96 opposed to the first transverse direction end edge 94, and a pair of opposing longitudinal direction side edges, 98 and 100, extending between and connecting the first transverse direction end edge 94 and the second transverse direction end edge 96. The perimeter edge 92 defines the overall shape of the absorbent core 56. In various embodiments, the perimeter edge 92 defines a shape of an absorbent core 56 which is any shape as deemed suitable for the absorbent article 10. In various embodiments, the absorbent core 56 can have a shape providing the absorbent core 56 with a uniform transverse direction width through the longitudinal direction length of the absorbent core 56. In various embodiments, the absorbent core 56 can have a shape providing the absorbent core 56 with a non-uniform transverse direction width through the longitudinal direction length of the absorbent core 56. In various embodiments, the absorbent core 56 can have a first portion located within the crotch region 40, a second portion located within a portion of the first waist region 20, and a third portion located within a portion of the second waist region 30. In such embodiments, the second portion of the absorbent core 56 can have a maximum transverse direction width 114 in the first waist region 20 and can have a maximum transverse direction width 116 in the second waist region 30. In various of such embodiments, the maximum transverse direction width 114 of the absorbent core 56 in the first waist region 20 can be the same as the maximum transverse direction width 116 of the absorbent core 56 in the second waist region 30. In various of such embodiments, the maximum transverse direction width 114 of the absorbent core 56 in the first waist region 20 can be greater than the maximum transverse direction width 116 of the absorbent core 56 in the second waist region 30. In various of such embodiments, the maximum transverse direction width 114 of the absorbent core 56 in the first waist region 20 can be less than the maximum transverse direction width 116 of the absorbent core 56 in the second waist region 30. The absorbent core 56 can have a topsheet layer facing surface 110 and a backsheet layer facing surface 112.

The absorbent core 56 can have an absorbent core midpoint 102 which is the location halfway between the first transverse direction end edge 94 of the absorbent core 56 and the second transverse direction end edge 96 of the absorbent core 56. The absorbent core midpoint 102 is positioned within the crotch region 40 of the absorbent article 10 and between the absorbent article narrowest width 80 and the transverse axis 14 of the absorbent article 10. The absorbent core midpoint 102, therefore, does not coincide with either the absorbent article narrowest width 80 or the transverse axis 14 of the absorbent article 10. In various embodiments, the absorbent core midpoint 102 is offset from the absorbent article narrowest width 80 by a distance 104 in the longitudinal direction (X) of at least 10, 12, 14, 16, 18, 20, 22, 24, 26, 28, or 30 mm. In various embodiments, the absorbent core midpoint 102 is offset from the absorbent article narrowest width 80 by a distance 104 in the longitudinal direction (X) from about 10, 12, 14, 16, or 18 mm, to about 20, 22, 24, 26, 28, or 30 mm. In various embodiments, the absorbent core midpoint 102 is offset from the absorbent article narrowest width 80 by a distance 104 in the longitudinal direction (X) of at least 1% of the absorbent article 10 total article length 70. In various embodiments, the absorbent core midpoint 102 is offset from the absorbent article narrowest width 80 by a distance 104 in the longitudinal direction (X) from about 1 or 2% to about 4 or 5% of the absorbent article 10 total article length 70. In various embodiments, the absorbent core midpoint 102 is offset from the transverse axis 14 of the absorbent article 10 by a distance 106 in the longitudinal direction (X) of at least 50, 52, 54, 56, 58, 60, 62, 64, 66, 68, 70, 72, 74, or 76 mm. In various embodiments, the absorbent core midpoint 102 is offset from the transverse axis 14 of the absorbent article 10 by a distance 106 in the longitudinal direction (X) from about 50, 52, 54, 56, 58, 60, or 62 mm to about 64, 66, 68, 70, 72, 74, or 76 mm. In various embodiments, the absorbent core midpoint 102 is offset from the transverse axis 14 of the absorbent article 10 by a distance 106 in the longitudinal direction (X) of from about 6 or 8% to about 9 or 15% of the absorbent article 10 total article length 70. As described herein, an absorbent article 10 having a more rectangular shape within the crotch region 40 may provide too much absorbent article bulk between the legs of the wearer of the absorbent article 10 which can result in the absorbent article bunching up between the wearer's legs, protruding away from the body of the wearer and not fitting close to the body of the wearer. Additionally, wearers of absorbent articles have body shapes which are a variety of shapes, sizes, and curvature, and generally not symmetrical. Positioning, the absorbent article narrowest width 80 closer to the first waist edge 22 can provide for a non-symmetrical absorbent article 10 wherein a greater proportion of the absorbent article 10 is positioned on the posterior side of the wearer's body providing for better coverage of the buttocks of the wearer of the absorbent article 10 and a lesser proportion of the absorbent article 10 is positioned on the anterior side of the wearer's body providing for a reduction of friction between the material of the absorbent article 10 and the wearer's legs when they move. Positioning the absorbent core midpoint 102 between the absorbent article narrowest width 80 and the transverse axis 14 can place the absorbent core 56 where it is needed most for capture of body exudate released from the wearer of the absorbent article 10 and minimize the bulkiness of the material of the absorbent core 56 between the legs of the wearer of the absorbent article 10.

By way of example, suitable materials and/or structures for the absorbent core 56 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al. each of which is hereby incorporated by reference thereto in its entirety.

In various embodiments, an absorbent core 56 can be a single layer structure and can include, for example, a matrix of cellulosic fluff and superabsorbent material. In various embodiments, an absorbent core 56 can have at least two layers of material, such as, for example, a body facing layer and a garment facing layer. In various embodiments, the two layers can be identical to each other. In various embodiments, the two layers can be different from each other. In such embodiments, the two layers can provide the absorbent article 10 with different absorption properties as deemed suitable. In various embodiments, the body facing layer of the absorbent core 56 may be constructed of an airlaid material and the garment facing layer of the absorbent core 56 may be constructed of a superabsorbent polymer-containing compressed sheet. In such embodiments, the airlaid material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm.

The absorbent core 56 can be at least partially enveloped by a core wrap 200. The core wrap 200 can be utilized to maintain the structural integrity of the absorbent core 56. For example, the absorbent core 56 may contain superabsorbent material and the core wrap 200 can be utilized to maintain the superabsorbent material within the absorbent core 56.

In various embodiments, the core wrap 200 can be formed from a single component of material which has, in a laid flat configuration, a transverse direction width dimension larger than the maximum transverse direction width dimension of the absorbent core 56. In various embodiments, the core wrap 200 can be formed from two or more components of material wherein, when in a laid flat configuration, the two or more components are generally in a side-by-side configuration with some or no overlap of the two or more components to form the core wrap 200 and the core wrap 200 has a transverse direction width dimension larger than the maximum transverse direction width dimension of the absorbent core 56. In various embodiments, to at least partially envelop the absorbent core 56, a portion of the core wrap 200 is positioned in a face-to-face relationship with the backsheet layer facing surface 112 of the absorbent core 56 and portions of the core wrap 200 extending transversely beyond the absorbent core 56 are folded over the absorbent core 56 and placed into a face-to-face relationship with the topsheet layer facing surface 110 of the absorbent core 56. In various embodiments, less than the entire topsheet layer facing surface 110 of the absorbent core 56 is in a face-to-face relationship with the core wrap 200. In various embodiments, the entire topsheet layer facing surface 110 of the absorbent core 56 is in a face-to-face relationship with the absorbent core 56. In various embodiments, to at least partially envelope the absorbent core 56, a portion of the core wrap 200 is positioned in a face-to-face relationship with the topsheet layer facing surface 110 of the absorbent core 56 and portions of the core wrap 200 extending transversely beyond the absorbent core 56 are folded over the absorbent core 56 and placed into a face-to-face relationship with the backsheet layer facing surface 112 of the absorbent core 56. For reasons described herein, in such embodiments, the entire backsheet layer facing surface 112 of the absorbent core 56 is in a face-to-face relationship with the core wrap 200. In the Figures, the exemplary illustrations of the core wrap 200 illustrate a configuration of the core wrap 200 wherein the entire topsheet layer facing surface 110 of the absorbent core 56 is in a face-to-face relationship with the core wrap 200, the entire backsheet layer facing surface 112 of the absorbent core 56 is in a face-to-face relationship with the core wrap 200, and the core wrap 200 overlaps itself on the backsheet layer facing surface 112 of the absorbent core 56 to form a lap seam 202. While a lap seam 202 is illustrated on the backsheet layer facing surface 112 of the absorbent core 56, alternate types of seams such as, for example, a butt seam or flange seam may be suitable and may be positioned, in the alternative, on the topsheet layer facing surface 110 of the absorbent core 56.

In various embodiments, the core wrap 200 can have, in a laid flat configuration, a longitudinal direction length dimension that is the same as or greater than the maximum longitudinal direction length dimension of the absorbent core 56. In the Figures, the exemplary illustrations of the core wrap 200 illustrate a configuration of the core wrap 200 which has a longitudinal direction length dimension which is the same as the longitudinal direction length dimension of the absorbent core 56.

The core wrap 200 can be pliable, less hydrophilic than the absorbent core 56, and sufficiently porous to be liquid permeable to thereby permit liquid to readily penetrate through its thickness to the absorbent core 56. The core wrap 200 also has sufficient structural integrity to withstand wetting thereof and of the absorbent core 56. The core wrap 200 can be constructed of a single layer of material or it may be a laminate constructed of two or more layers of material.

In various embodiments, the core wrap 200 can be constructed of one or more layers of a nonwoven web. In various embodiments, the nonwoven web can be constructed, at least in part, of a thermoplastic material. In various embodiments, the nonwoven web can be a meltblown web. In various embodiments, the nonwoven web can be a laminate constructed of a meltblown web laminated to at least one, or between two, spunbond nonwoven layers (e.g., a spunbond-meltblown web or a spunbond-meltblown-spunbond web). In various embodiments, the core wrap 200 can be constructed from a tissue web. In various embodiments, the core wrap 200 can be constructed as a film layer having apertures formed therein. In various embodiments, the core wrap 200 can be constructed from any materials deemed suitable for forming the topsheet layer 52 or the backsheet layer 54.

In various embodiments, the absorbent assembly 50 can include a surge layer 58 positioned between the absorbent core 56 and the topsheet layer 52. The surge layer 58 can be adapted to work with the absorbent core 56 in absorbing body exudates. In various embodiments, the surge layer 58 can have a higher void volume than the absorbent core 56 to quickly intake and hold body exudates so that the absorbent core 56 has time to absorb the body exudates without such body exudates leaking from the absorbent article 10. The surge layer 58 can take on any size and shape as desired and as deemed suitable. For example, in FIG. 3, the surge layer 58 is illustrated in the shape of a rectangle and has a size dimension smaller than the absorbent core 56.

The absorbent assembly 50 can have a first transverse direction end edge 120, a second transverse direction end edge 122 opposed to the first transverse direction end edge 120, and a pair of opposing longitudinal direction side edges, 124 and 126, extending between and connecting the first transverse direction end edge 120 and the second transverse direction end edge 122. In various embodiments, such as illustrated in FIGS. 1-3, the non-linear portions of the longitudinal direction side edges, 124 and 126, of the absorbent assembly 50 can be arcuate and can form portions of the first leg side edge 82 and the second leg side edge 84, respectively, of the absorbent article 10.

The absorbent article 10 can have longitudinally extending elastic material 130 wherein a portion of the elastic material 130 is located at least within the crotch region 40 of the absorbent article 10 and positioned between each of the first longitudinal direction side edge 98 of the absorbent core 56 and the first leg side edge 82 of the absorbent article 10 and the second longitudinal direction side edge 100 of the absorbent core 56 and the second leg side edge 84 of the absorbent article 10. Each elastic material 130 can be an elastic strand, ribbon, or strip of elastic material. For example, with reference to FIG. 3, the elastic material 130 is a plurality of elastic strands extending longitudinally between the first longitudinal direction side edge 98 of the absorbent core 56 and the first leg side edge 82 of the absorbent article 10 and a plurality of elastic strands forming the elastic material 130 extending longitudinally between the second longitudinal direction side edge 100 of the absorbent core 56 and the second leg side edge 84 of the absorbent article 10. While the elastic material 130 is illustrated as longitudinally extending from the first waist region 20, through the crotch region 40, and to the second waist region 30, it is to be understood that the elastic material 130 can be positioned in only the crotch region 40, in a combination of the crotch region 40 and first waist region 20, in a combination of the crotch region 40 and second waist region 30, or in a combination, such as illustrated, of the crotch region 40, first waist region 20, and second waist region 30.

Each of the longitudinally extending elastic materials 130 can have an interior perimeter which is the portion of the elastic materials 130 closest to the absorbent core 56 without coming into a configuration such that it will overlay the absorbent core 56. The interior perimeter of each of the longitudinally extending elastic materials 130 can be positioned at a spatial distance 132 from the longitudinal direction side edges, 98 and 100, of the absorbent core 56. The spatial distance 132 of each of the longitudinally extending elastic materials 130 from each of the longitudinal direction side edges, 98 and 100, at the locations of each of the absorbent article narrowest width 80 and the transverse axis 14 can be a minimum of 15 mm. In various embodiments, the spatial distance 132 at each of the locations of the absorbent article narrowest width 80 and the transverse axis 14 can be from about 15, 17, 19, or 21 mm to about 23, 25, 27, 29, or 31 mm. In various embodiments, the spatial distance 132 at each of the locations of the absorbent article narrowest width 80 and the transverse axis 14 can be a minimum of 1.5, 2, 2.5, 3, 3.5, 4, 4.5, 5, or 5.5% of the first waist region width 28. In various embodiments, the spatial distance 132 can be uniform. In various embodiments, the spatial distance 132 can be non-uniform. Such a spatial distance 132 can allow for movement of the absorbent core 56 as the wearer moves their body. The spatial distance 132 can isolate the absorbent core 56 of the absorbent article 10 such that when the wearer of the absorbent article 10 moves their body and/or legs, the absorbent core 56 is not impacted by such movement of the wearer. If the longitudinally extending elastic materials 130 were not separated by a minimum spatial distance 132 from the absorbent core 56, the movement of the wearer could cause the elastic materials 130 to pull and twist the absorbent core 56 which can lead to movement of the absorbent core 56 out of proper placement for capturing body exudates. In various embodiments, the longitudinally extending elastic materials 130 can be non-linear or linear as they extend in the longitudinal direction (X) of the absorbent article 10.

As illustrated in FIGS. 1-4, the first waist region 20 of the absorbent article 10 can be constructed of an elastomeric panel 140. The elastomeric panel 140 of the first waist region 20 can be bordered by a lower edge 142, the first longitudinal direction side edge 24, the second longitudinal direction side edge 26, and the first waist edge 22. The second waist region 30 can be constructed of an elastomeric panel 150. The elastomeric panel 150 of the second waist region 30 can be bordered by a lower edge 152, the first longitudinal direction side edge 34, the second longitudinal direction side edge 36, and the second waist edge 32. The elastomeric panels, 140 and 150, can have elastic material, 144 and 154, respectively, such that the elastomeric panels, 140 and/or 150, upon application of a stretching force, are stretchable in the transverse direction (Y), and which upon release of the stretching force, contracts/returns at least a portion of its stretched length, desirably to its original dimension.

In various embodiments, the elastic material, 144 and 154, in each of the elastomeric panels, 140 and 150, can be elastomeric strands of material such as can be preformed from LYCRA brand fibers/yarns for example. LYCRA is a registered trademark of E.I. DuPont DeNemours Co., Wilmington, Del., U.S.A. The elastomeric strands can have a round, semi-circular, square, rectangular, oval, or other geometrical configuration. In various embodiments, the plurality of elastomeric strands can be elastomeric in at least the transverse direction of the absorbent article 10 and can extend from the first longitudinal direction side edge, 24 or 34, to the second longitudinal direction side edge, 26 or 36, of the elastomeric panels, 140 or 150, respectively. When present, the elastomeric strands can be positioned in a longitudinal direction spaced apart configuration. In various embodiments, the elastic material, 144 and 154, can be an elastomeric polymeric film layer. The elastomeric polymeric film layer can be elastomeric in at least the transverse direction of the absorbent article 10 and can extend from the first longitudinal direction side edge, 24 or 34, to the second longitudinal direction side edge, 26 or 36, of the elastomeric panels, 140 or 150, respectively. In various embodiments, a suitable elastomeric polymeric film layer can be a stretch-bonded laminate (SBL) in which an elastic core or middle layer is elongated before two opposing outer nonwoven web layers are bonded thereto. Another suitable material for the elastomeric polymeric film layer is a necked bonded laminate (NBL). The NBL material is a three layer laminate but the elastic core or middle layer is not pre-stretched prior to being attached to the two outer nonwoven web layers. Instead, the opposing outer nonwoven web layers are necked stretched before the elastic core or middle layer is bonded to them. Other examples of such elastomeric materials that can be used as an elastomeric polymeric film layer include a continuous filament stretch bonded laminate (CFSBL), a vertical filament laminate (VFL), a necked stretch bonded laminate (NSBL), or a necked thermal laminate (NTL). Combinations of such materials can also be used. Such materials are described in U.S. Pat. No. 4,720,415 to Vander Wielen et al., U.S. Pat. No. 5,366,793 to Fitts, et al., U.S. Pat. No. 5,385,775, to Wright, U.S. Pat. No. 6,969,441 to Welch et al., U.S. Pat. No. 6,978,486 to Zhou et al., U.S. Pat. No. 7,803,244 to Siqueira et al., and U.S. Pat. No. 5,226,992 to Morman et al., each of which are hereby incorporated by reference thereto in its entirety. The elastomeric laminates just described will typically include an elastomeric layer and at least one surface-bonded nonwoven web layer such as a meltblown, spunbond, or through-air bonded web.

To form each of the elastomeric panels, 140 and 150, the elastic material, 144 and 154, within the elastomeric panels, 140 and 150, can be sandwiched between a single nonwoven material which has been folded over onto itself or can be sandwiched between two separate nonwoven materials. For example, as illustrated in FIGS. 3 and 4, each of the elastomeric panels, 140 and 150, are formed by sandwiching an elastic material, 144 and 154, such as, for example, a polymeric film sheet, between a pair of nonwoven materials, 146 and 148, in the first waist region 20 and a pair of nonwoven materials, 156 and 158, in the second waist region 30. The elastic materials, 144 and 154, can be sandwiched and held between the nonwoven layers, 146, 148, 156, and 158, with adhesive, ultrasonic bonding, heat pressure sealing, or any other means deemed suitable.

Each of the elastomeric panels, 140 and 150, can have a first portion, 160 and 170, respectively, which can be the waist portion of the absorbent article 10. In various embodiments, each first portion, 160 and 170, of the elastomeric panels, 140 and 150, respectively, can have a length in the longitudinal direction (X) which is less than about 5, 4, or 3% of the absorbent article length 70. In the illustrative and exemplary embodiments of FIGS. 1-4, the first portion, 160 and 170, can be further illustrated as having a waist edge, 22 and 32, formed by folding a portion of the elastomeric panels, 140 and 150, onto itself creating a fold, 164 and 174, and placing a material edge, 166 and 176, on the exterior of the absorbent article 10. In various embodiments, the elastic material, 144 and 154, in each of the first portions, 160 and 170, of the elastomeric panels, 140 and 150, can have a uniform tension in the transverse direction (Y) and in the longitudinal direction (X). To create additional tension in the first portions, 160 and 170, of the elastomeric panels, 140 and 150, it may desirable to include secondary elastic material, such as, for example, elastic strands 180, within the fold, 164 and 174.

Each of the elastomeric panels, 140 and 150, can have a second portion, 162 and 172, respectively, which can be the chassis portion of the absorbent article 10. The second portion, 162 and 172, of each of the elastomeric panels, 140 and 150, respectively, can exclude the first portions, 160 and 170, and can extend in the longitudinal direction (X) from the first portions, 160 and 170, towards the crotch region 40 of the absorbent article 10. As described herein, in various embodiments, the absorbent core 56 can have a first portion in the crotch region 40 of the absorbent article 10 and another portion located in a portion of the second portion, 162 or 172, of at least one of the first waist region 20 or the second waist region 30. In such embodiments, the second portion of the absorbent core 56 located in a second portion, 162 or 172, of at least one of the first waist region 20 or the second waist region 30 can be in an overlapping configuration with a portion of the elastic material, 144 or 154, in the respective second portion, 162 or 172, of the first waist region 20 and/or the second waist region 30. In various embodiments, the absorbent core 56 can have a first portion located in the crotch region 40, a second portion located in a portion of the second portion 162 of the first waist region 20, and a third portion located in a portion of the second portion 172 of the second waist region 30. In such embodiments, the second portion of the absorbent core 56 located in the second portion 162 of the first waist region 20 can be in an overlapping configuration with a portion of the elastic material 144 in the second portion 162 of the first waist region 20 and the third portion of the absorbent core 56 located in the second portion 172 of the second waist region 30 can be in an overlapping configuration with a portion of the elastic material 154 in the second portion 172 of the second waist region 30.

The elastic material 144 forming the elastomeric panel 140 of the first waist region 20 and the elastic material 154 forming the elastomeric panel 150 of the second waist region 30 are elastomeric in at least the transverse direction (Y) of the absorbent article 10. The elastic materials, 144 and 154, are elastomeric in that they can be elongated by at least 50 percent of their relaxed length and which will recover, upon release of the applied force, at least 50 percent of their elongation. The elastic material, 144 and 154, in the first waist region 20 and the second waist region 30, respectively, are active and capable of stretching/retracting continuously in the transverse direction (Y).

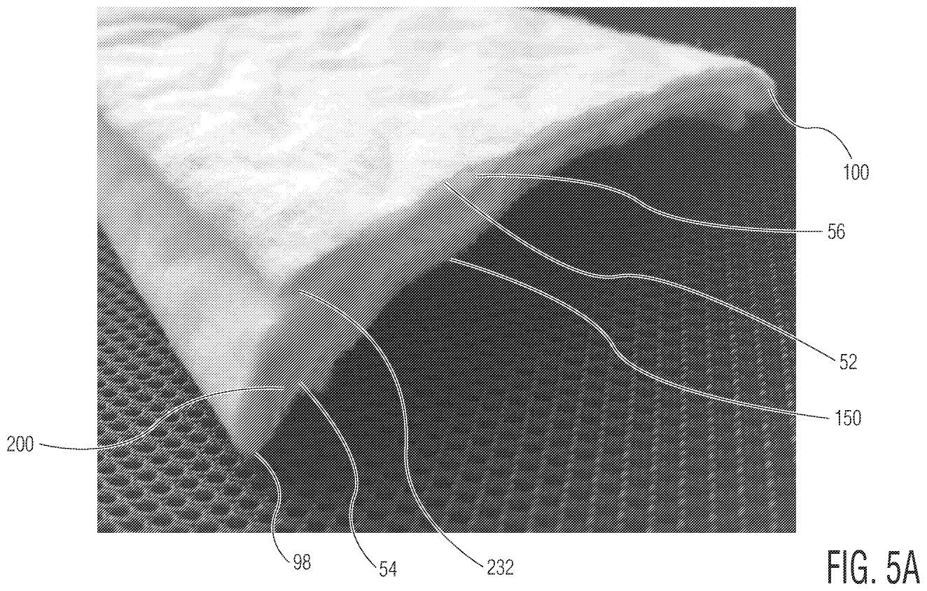

In a relaxed configuration, the elastic material, 144 and 154, of each of the first waist region 20 and the second waist region 30 can cause the material forming the first waist region 20 and the second waist region 30 to fold and bunch up similar to an accordion in the transverse direction (Y) of the absorbent article 10. In the formation of the absorbent article 10, the backsheet layer 54 of the absorbent assembly 50 is bonded to the material forming the first waist region 20 and the second waist region 30. In various embodiments, the elastic materials, 144 and/or 154, may also impact the layers of the absorbent assembly 50 and their ability to remain in a generally planar configuration. FIGS. 5A and 5B are photographs of portions of an absorbent article 10 in a relaxed configuration. FIG. 5A is a perspective view of the portion of the absorbent article 10 and FIG. 5B is an end view of the same portion of the absorbent article 10. The absorbent article 10 of FIGS. 5A and 5B was formed as an absorbent assembly 50 bonded to elastomeric panels, 140 and 150, forming a first waist region 20 and a second waist region 30, respectively. The absorbent assembly 50 had an absorbent core 56 at least partially enveloped by a core wrap 200 and sandwiched between a topsheet layer 52 and a backsheet layer 54. A single material forming the core wrap 200 was positioned with a portion of the core wrap 200 in a face-to-face and bonded relationship with the entire topsheet layer facing surface 110 of the absorbent core 56 and portions of the core wrap 200 were then folded over the absorbent core 56 such that the entire backsheet layer facing surface 112 of the absorbent core 56 was also in a face-to-face relationship with the core wrap 200. The portion of the core wrap 200 which was in a face-to-face relationship with the backsheet layer facing surface 112 of the absorbent core 56 was bonded to the backsheet layer 54 in order to maintain the proper placement of the absorbent core 56 within the absorbent article 10. This same portion of the core wrap 200, however, was not bonded to the backsheet layer facing surface 112 of the absorbent core 56 and could shift its positioning relative to the backsheet layer facing surface 112 of the absorbent core 56. A surge layer 58 (not visible in the photographs) was further positioned between the core wrapped absorbent core 56 and the topsheet layer 52. A portion of the backsheet layer 54 of the absorbent assembly 50 was bonded to a portion of an elastomeric panel 140 forming the first waist region 20 and a portion of the backsheet layer 54 of the absorbent assembly 50 was bonded to a portion of the elastomeric panel 150 forming the second waist region 30. A first portion of the core wrapped absorbent core 56 was located in the crotch region 40, a second portion of the core wrapped absorbent core 56 was located in a portion of the first waist region 20, and a third portion of the core wrapped absorbent core 56 was located in a portion of the second waist region 30. The absorbent core 56 had a shape that was non-uniform in the longitudinal direction of the absorbent core 56 but was symmetrical about a transverse axis of the absorbent core 56 and had a first maximum transverse direction width 114 in the first waist region 20 and had a second maximum transverse direction width 116 in the second waist region 30. The first maximum transverse direction width 114 was the same as the second maximum transverse direction width 116. The core wrap 200 was in an abutting and face-to-face but non-bonded relationship with each of the longitudinal direction side edges, 98 and 100, of the absorbent core 56 at the first maximum direction transverse width 114 of the absorbent core 56 in the first waist region 20 and at the second maximum direction transverse width 116 of the absorbent core 56 in the second waist region 30. The bonding of the absorbent assembly 50 to each of the first waist region 20 and second waist region 30 occurred when each of the first waist region 20 and second waist region 30 were in a transverse direction and longitudinal direction fully extended configuration. Each of the elastomeric panels, 140 and 150, were formed of an elastic film material sandwiched between two nonwoven layers. The absorbent article 10 was cross-sectioned in the transverse direction (Y) through an overlapping region wherein the core wrapped absorbent core 56 overlaps a portion of the elastomeric panel 150 forming the second waist region 30. As is visible in each of FIGS. 5A and 5B, when the absorbent article 10 was allowed to assume a relaxed configuration (and not the assembly configuration of a fully extended configuration), the elastic material of the elastomeric panel 150 folded and bunched up which further caused the elastomeric panel 150, the backsheet layer 54, and the portion of the core wrap 200 which was in a face-to-face but non-bonded relationship with the backsheet layer facing surface 112 of the absorbent core 56 to fold and bunch up similar to an accordion. As the portion of the core wrap 200 in a face-to-face relationship with the backsheet layer facing surface 112 of the absorbent core 56 folded and bunched up it pulled, in the transverse direction, the remainder of the core wrap 112 enveloping the absorbent core 56, such as the portions of the core wrap 200 in an abutting and face-to-face, but non-bonded, relationship with the longitudinal direction side edges, 98 and 100, of the absorbent core 56 and the portion of the core wrap 200 in a face-to-face and bonded relationship with the topsheet layer facing surface 110 of the absorbent core 56, in a direction towards the longitudinal axis 12 of the absorbent article 10. The pulling of one portion of the core wrap 200 on the remaining core wrap 200 resulted in the absorbent core 56 buckling and folding over on itself such as is visible with fold 232. The buckling and folding of the absorbent core 56 can compromise the overall comfort, discretion, and effectiveness of the absorbent article 10. The wearer of the absorbent article 10 may experience discomfort when wearing the absorbent article 10 and may experience an increase in anxiety if they believe the effectiveness of the absorbent article 10 has been diminished or if they feel the product may be more readily discerned through their clothing by another.

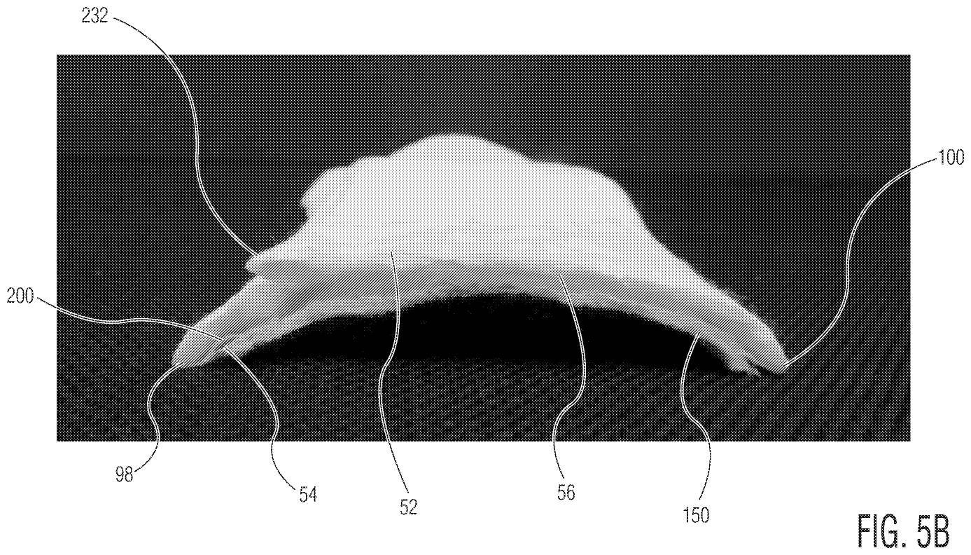

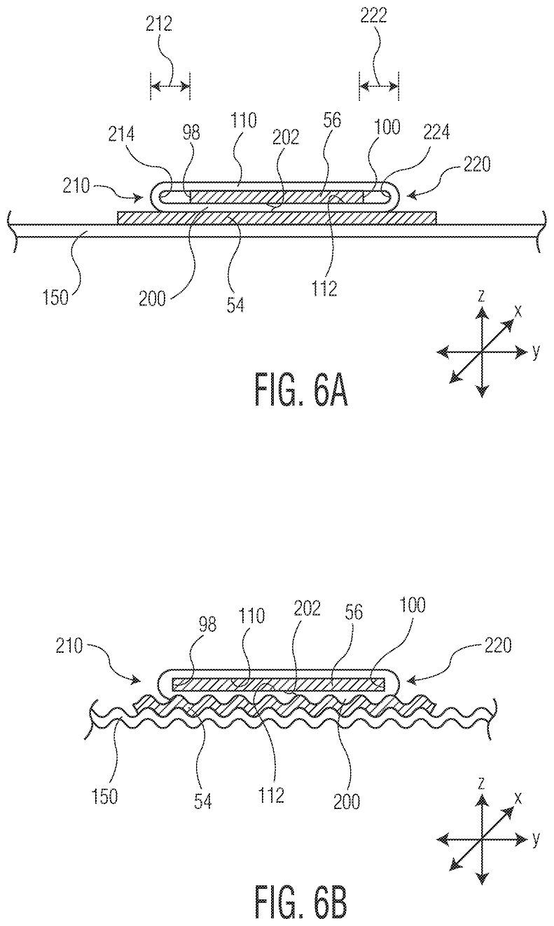

Referring to FIGS. 6A and 6B, an embodiment of an absorbent article 10 construction is illustrated wherein the overall comfort, discretion, and effectiveness of the absorbent article 10 can be maintained for the wearer of the absorbent article 10. FIG. 6A is a cross-sectional illustration of a portion of an absorbent article 10 in a transversely stretched and laid flat configuration while FIG. 6B is a cross-sectional illustration of the portion of the absorbent article 10 of FIG. 6A in a traverse direction (Y) relaxed configuration. The portion of the absorbent article 10 illustrated in FIGS. 6A and 6B includes an absorbent core 56 at least partially enveloped by a core wrap 200. A single material forming the core wrap 200 is positioned with a portion of the core wrap 200 in a face-to-face and bonded relationship with the entire topsheet layer facing surface 110 of the absorbent core 56 and portions of the core wrap 200 then folded over the absorbent core 56 such that the entire backsheet layer facing surface 112 of the absorbent core 56 is also in a face-to-face relationship with the core wrap 200. When folding the core wrap 200 from the topsheet layer facing surface 110 to the backsheet layer facing surface 112 of the absorbent core 56, the core wrap is maintained in a spaced apart configuration from the longitudinal direction side edges, 98 and 100, of the absorbent core 56. Thus, the core wrap 200 can have a first side 210 which has an interior surface 214 that is spaced apart from the longitudinal direction side edge 98 of the absorbent core 56 by a first distance 212. The core wrap 200 has a second side 220 which has an interior surface 224 that is spaced apart from the longitudinal direction side edge 100 of the absorbent core by a second distance 222. The first distance 212 and the second distance 222 separating the interior surface, 214 and 224, of the core wrap 200 from the longitudinal direction side edges, 98 and 100, is at least about 3.5, 4, 4.5, 5, 5.5, 6, 6.5, 7, 7.5, 8, 8.5, 9, 9.5, or 10 mm on each side of the absorbent core 56 when the absorbent article 10 is in a transverse direction fully extended configuration. The portion of the core wrap 200 in a face-to-face relationship with the backsheet layer facing surface 112 of the absorbent core 56 is bonded to the backsheet layer 54 in order to maintain the proper placement of the absorbent core 56 within the absorbent article 10. This same portion of the core wrap 200, however, is not bonded to the backsheet layer facing surface 112 of the absorbent core 56 and maintains its ability to shift its positioning relative to the backsheet layer facing surface 112 of the absorbent core 56. A portion of the backsheet layer 54 of the absorbent assembly 50 is bonded to a portion of an elastomeric panel, 140 and/or 150, forming a waist region, 20 and/or 30. For illustration within FIGS. 6A and 6B, the backsheet layer 54 of the absorbent assembly 50 is bonded to a portion of the elastomeric panel 150 of the second waist region 30, thereby illustrating a portion of a core wrapped absorbent core 56 in an overlapping configuration with elastic material sandwiched between two nonwoven materials. The bonding of the backsheet layer 54 of the absorbent assembly 50 to the elastomeric panel 150 occurs when the elastomeric panel 150 is in a transverse direction fully extended configuration such as illustrated in FIG. 6A. When the absorbent article 10 transitions from a transverse direction fully extended configuration such as illustrated in FIG. 6A to a relaxed configuration such as illustrated in FIG. 6B, the elastic material of the elastomeric panel 150 can fold and bunch up and can cause the elastomeric panel 150, the backsheet layer 54, and the portion of the core wrap 200 which is in a face-to-face but non-bonded relationship with the backsheet layer facing surface 112 of the absorbent core 56 to fold and bunch up similar to an accordion. However, unlike FIGS. 5A and 5B, the excess material of the core wrap 200, which is separated from the longitudinal direction side edges, 98 and 100, of the absorbent core 56 by a distance 212 and 222, respectively, on each side of the absorbent core 56, can absorb the tension of the elastic material of the elastomeric panel 150 and the pulling, in the transverse direction, by the portion of the core wrap 200 which is in a face-to-face and non-bonded relationship with the backsheet layer facing surface 112 of the absorbent core 56 in a direction towards the longitudinal axis 12 of the absorbent article 10. It is the excess material of the core wrap 200 which can fold and bunch up thereby leaving the absorbent core 56 in a generally flat and planar configuration.

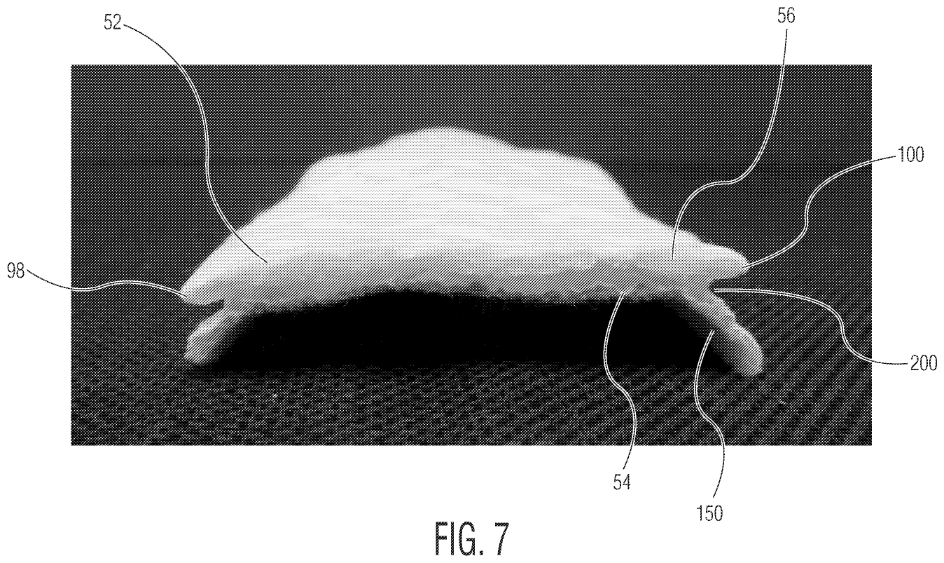

FIG. 7 is a photograph of a portion of an absorbent article 10 in a relaxed configuration demonstrating this movement, described with regard to FIGS. 6A and 6B, of the material layers of the absorbent article 10. The absorbent article 10 of FIG. 7 was formed as an absorbent assembly 50 bonded to elastomeric panels, 140 and 150, forming a first waist region 20 and a second waist region 30, respectively. The absorbent assembly 50 had an absorbent core 56 at least partially enveloped by a core wrap 200 and sandwiched between a topsheet layer 52 and a backsheet layer 54. A single material forming the core wrap 200 was positioned with a portion of the core wrap 200 in a face-to-face and bonded relationship with the entire topsheet layer facing surface 110 of the absorbent core 56 and portions of the core wrap 200 were then folded over the absorbent core 56 such that the entire backsheet layer facing surface 112 of the absorbent core 56 was also in a face-to-face relationship with the core wrap 200. The portion of the core wrap 200 which was in a face-to-face relationship with the backsheet layer facing surface 112 of the absorbent core 56 was bonded to the backsheet layer 54 in order to maintain the proper placement of the absorbent core 56 within the absorbent article 10. This same portion of the core wrap 200, however, was not bonded to the backsheet layer facing surface 112 of the absorbent core 56 and could shift its positioning relative to the backsheet layer facing surface 112 of the absorbent core 56. A surge layer 58 (not visible in the photographs) was further positioned between the core wrapped absorbent core 56 and the topsheet layer 52. A portion of the backsheet layer 54 of the absorbent assembly 50 was bonded to a portion of an elastomeric panel 140 forming the first waist region 20 and a portion of the backsheet layer 54 of the absorbent assembly 50 was bonded to a portion of the elastomeric panel 150 forming the second waist region 30. A first portion of the core wrapped absorbent core 56 was located in the crotch region 40, a second portion of the core wrapped absorbent core 56 was located in a portion of the first waist region 20, and a third portion of the core wrapped absorbent core 56 was located in a portion of the second waist region 30. The absorbent core 56 had a shape that was non-symmetrical about a transverse axis of the absorbent core 56 and had a first maximum transverse direction width 114 in the first waist region 20 and had a second maximum transverse direction width 116 in the second waist region 30. The first maximum transverse direction width 114 was greater than the second maximum transverse direction width 116. The core wrap 200 was in an abutting and face-to-face but non-bonded relationship with each of the longitudinal direction side edges, 98 and 100, of the absorbent core 56 at the first maximum transverse direction width 114 of the absorbent core 56 in the first waist region 20. The core wrap 200 was spaced apart from each of the longitudinal direction side edges, 98 and 100, of the absorbent core 56 at the second maximum transverse direction width 116 by 3.5 mm on each side of the absorbent core 56. The bonding of the absorbent assembly 50 to each of the first waist region 20 and second waist region 30 occurred when each of the first waist region 20 and second waist region 30 were in a fully extended configuration. Each of the elastomeric panels, 140 and 150, were formed of an elastic film material sandwiched between two nonwoven layers. The absorbent article 10 was cross-sectioned in the transverse direction (Y) through an overlapping region wherein the core wrapped absorbent core 56 overlaps a portion of the elastomeric panel 150 forming the second waist region 30. As is visible in FIG. 7, when the absorbent article 10 was allowed to assume a relaxed configuration (and not the assembly configuration of a fully extended configuration), the elastic material of the elastomeric panel 150 folded and bunched up which further caused the elastomeric panel 150, the backsheet layer 54, and the portion of the core wrap 200 which was in a face-to-face but non-bonded relationship with the backsheet layer facing surface 112 of the absorbent core 56 to fold and bunch up similar to an accordion. As the portion of the core wrap 200 in a face-to-face and non-bonded relationship with the backsheet layer facing surface 112 of the absorbent core 56 folded and bunched up it pulled, in a transverse direction (Y), the remainder of the core wrap 112 enveloping the absorbent core 56, such as the portions of the core wrap 200 which were spaced apart from the longitudinal direction side edges, 98 and 100, at the second maximum transverse direction width 116 by 3.5 mm on each side of the absorbent core 56 and the portion of the core wrap 200 in a face-to-face and bonded relationship with the topsheet layer facing surface 110 of the absorbent core 56, in a direction towards the longitudinal axis 12 of the absorbent article 10. The pulling of one portion of the core wrap 200 on the remaining core wrap 200 resulted in the material of the core wrap 200 which was spaced apart from the longitudinal direction side edges, 98 and 100, of the absorbent core 56 at the second maximum transverse direction width 116 to move into an abutting relationship with each of the longitudinal direction side edges, 98 and 100, of the absorbent core 56, but not cause a buckling and folding over of the absorbent core 56 itself. The excess material of the core wrap 200 which was separated from the longitudinal direction side edges, 98 and 100, of the absorbent core 56 was able to absorb the tension of the elastic material of the elastomeric panel and the pulling, in the transverse direction, by the core wrap 200 in a direction towards the longitudinal axis 12 of the absorbent article 10. The excess material of the core wrap 200 was able to move into an abutting relationship with the longitudinal direction side edges, 98 and 100, of the absorbent core 56 and to fold and bunch up similar to an accordion on the backsheet layer facing surface 112 of the absorbent core 56 without compromising the comfort, discretion, and effectiveness of the absorbent article 10 as the absorbent core 56 was able to remain in a generally flat and planar configuration.

The core wrap 200 side edges, 210 and 220, can be in a spaced apart configuration from each of the longitudinal direction side edges, 98 and 100, of the absorbent core 56 by a distance of at least about 3.5, 4, 4.5, 5, 5.5, 6, 6.5, 7, 7.5, 8, 8.5, 9, 9.5, or 10 mm on each side of the absorbent core 56 in at least one of the first waist region 20 or the second waist region 30 when the absorbent article 10 is in a transverse direction laid flat and stretched configuration. In various embodiments, the core wrap 200 side edges, 210 and 220, can be in a spaced apart configuration from the longitudinal direction side edges, 98 and 100, of the absorbent core 56 in the first waist region 20 of the absorbent article 10 by at least 3.5 mm when the absorbent article 10 is in a transverse direction fully extended configuration. In such embodiments, the core wrap 200 side edges, 210 and 220, are spaced apart less than 3 mm from the longitudinal direction side edges, 98 and 100, of the absorbent core 56 in the second waist region 30 of the absorbent article 10 when the absorbent article is in a transverse direction fully extended configuration. In various embodiments, the core wrap 200 side edges, 210 and 220, can be in a spaced apart configuration from the longitudinal direction side edges, 98 and 100, of the absorbent core 56 in the second waist region 30 of the absorbent article 10 when the absorbent article 10 is in a transverse direction fully extended configuration. In such embodiments, the core wrap 200 side edges, 210 and 220, are spaced apart less than 3 mm from the longitudinal direction side edges, 98 and 100, of the absorbent core 56 in the first waist region 20 of the absorbent article 10 when the absorbent article is in a transverse direction fully extended configuration. In various embodiments in which the core wrap 200 side edges, 210 and 220, are spaced apart less than 3 mm from the longitudinal direction sides edges, 98 and 100, of the absorbent core 56 in either the first waist region 20 or the second waist region 30 of the absorbent article 10 when the absorbent article 10 is in a transverse direction fully extended configuration, the core wrap 200 side edges, 210 and 220, may abut the longitudinal direction side edges, 98 and 100, of the absorbent core 56.

In various embodiments, the core wrap 200 side edges, 210 and 220, can be in a spaced apart configuration from the longitudinal direction side edges, 98 and 100, of the absorbent core 56 in each of the first waist region 20 and the second waist region 30 of the absorbent article 10 when the absorbent article 10 is in a transverse direction fully extended configuration. In various embodiments wherein the core wrap 200 sides edges, 210 and 220, are spaced apart from the longitudinal direction side edges, 98 and 100, of the absorbent core 56 at each of the first maximum transverse direction width 114 and the second maximum transverse direction width 116, the core wrap 200 sides edges, 210 and 220, can be in a spaced apart configuration from each of the longitudinal direction side edges, 98 and 100, of the absorbent core 56 at each of the first maximum transverse direction width 114 and the second maximum transverse direction width 116 by the same distance with is at least 3.5 mm. In various embodiments wherein the core wrap 200 side edges, 210 and 220, are spaced apart from the longitudinal direction side edges, 98 and 100, of the absorbent core 56 at each of the first maximum transverse direction width 114 and the second maximum transverse direction width 116 by at least 3.5 mm, the core wrap 220 side edges, 210 and 220, can be in a spaced apart configuration from each of the longitudinal directions side edges, 98 and 100, of the absorbent core 56 at the first maximum transverse direction width 114 at a distance which is different from the distance spacing apart the core wrap 200 side edges, 210 and 220, from the longitudinal direction side edges, 98 and 100, at the second maximum transverse direction width. In various embodiments wherein the core wrap 200 side edges, 210 and 220, are spaced apart from the longitudinal direction side edges, 98 and 100, of the absorbent core 56 at each of the first maximum transverse direction width 114 and the second maximum transverse direction width 116 by at least 3.5 mm, the core wrap 220 side edges, 210 and 220, can be in a spaced apart configuration from each of the longitudinal directions side edges, 98 and 100, of the absorbent core 56 at the first maximum transverse direction width 114 at a distance which is greater than the distance spacing apart the core wrap 200 side edges, 210 and 220, from the longitudinal direction side edges, 98 and 100, at the second maximum transverse direction width 116. In various embodiments wherein the core wrap 200 side edges, 210 and 220, are spaced apart from the longitudinal direction side edges, 98 and 100, of the absorbent core 56 at each of the first maximum transverse direction width 114 and the second maximum transverse direction width 116 by at least 3.5 mm, the core wrap 220 side edges, 210 and 220, can be in a spaced apart configuration from each of the longitudinal directions side edges, 98 and 100, of the absorbent core 56 at the first maximum transverse direction width 114 at a distance which is less than the distance spacing apart the core wrap 200 side edges, 210 and 220, from the longitudinal direction side edges, 98 and 100, at the second maximum transverse direction width 116.

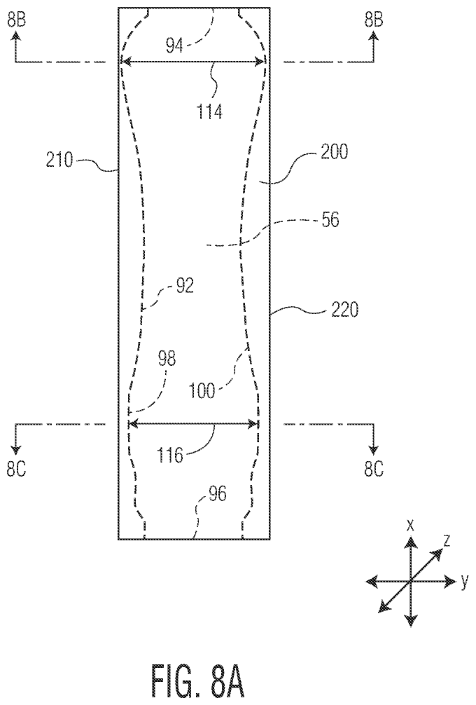

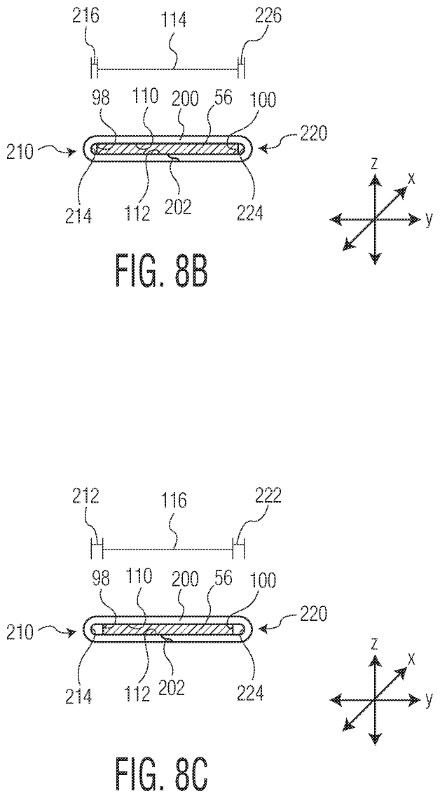

Referring to FIGS. 8A, 8B, and 8C, FIG. 8A is a top down view of an absorbent core 56 and core wrap 200, FIG. 8B is a cross-sectional view of the absorbent core 56 taken along line 8B-8B, and FIG. 8C is a cross-sectional view of the absorbent core 56 taken along line 8C-8C. As illustrated in FIGS. 8A, 8B, and 8C, the absorbent core 56 can be shaped so as to be non-symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is greater than the second maximum transverse direction width 116 of the absorbent core. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is less than 3 mm. The interior surfaces, 214 and 224, of the core wrap side edges, 210 and 220, respectively, can be spaced apart from the longitudinal direction side edges, 98 and 100, of the absorbent core at the second maximum transverse direction width 116 by a distance 212 and 222, respectively, which is at least 3.5 mm. In the exemplary embodiment illustrated in FIGS. 8A, 8B, and 8C, distances 212 and 222 are greater than distances 216 and 226.

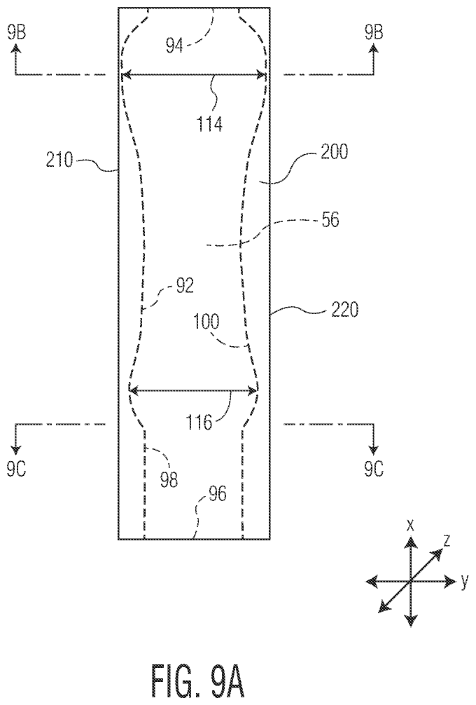

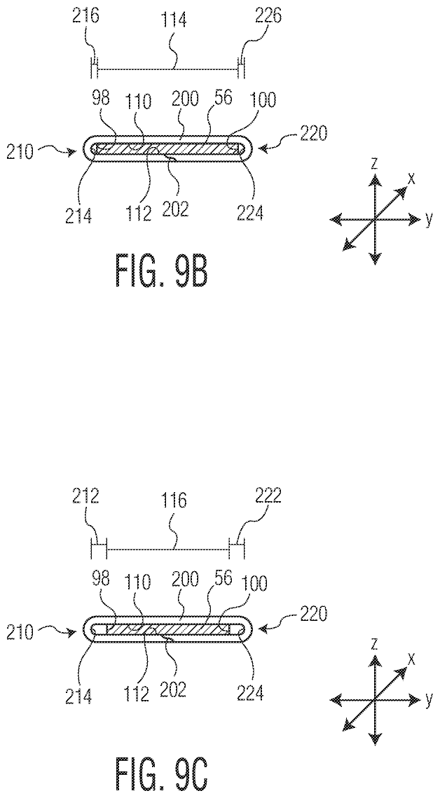

Referring to FIGS. 9A, 9B, and 9C, FIG. 9A is a top down view of an absorbent core 56 and core wrap 200, FIG. 9B is a cross-sectional view of the absorbent core 56 taken along line 9B-9B, and FIG. 9C is a cross-sectional view of the absorbent core 56 taken along line 9C-9C. As illustrated in FIGS. 9A, 9B, and 9C, the absorbent core 56 can be shaped so as to be non-symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is greater than the second maximum transverse direction width 116 of the absorbent core 56. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is less than 3 mm. The interior surfaces, 214 and 224, of the core wrap side edges, 210 and 220, respectively, can be spaced apart from the longitudinal direction side edges, 98 and 100, of the absorbent core at the second maximum transverse direction width 116 by a distance 212 and 222, respectively, which is at least 3.5 mm. In the exemplary embodiment illustrated in FIGS. 9A, 9B, and 9C, distances 212 and 222 are greater than distances 216 and 226.

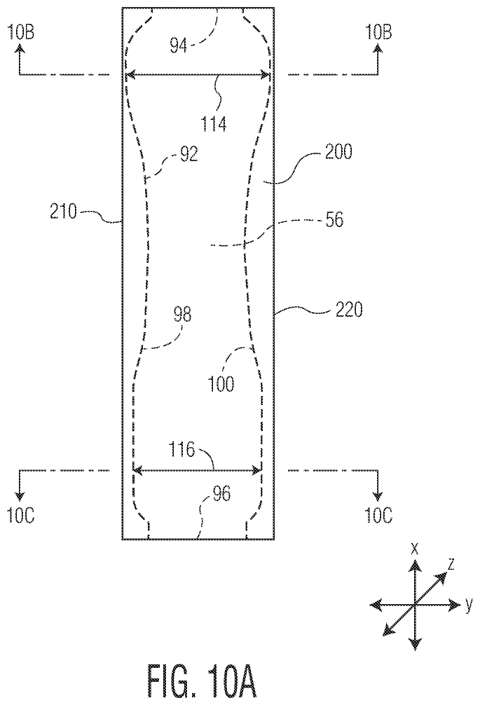

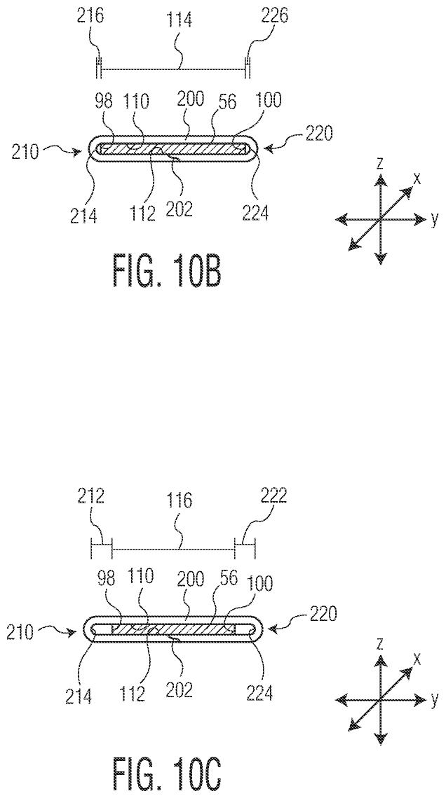

Referring to FIGS. 10A, 10B, and 100, FIG. 10A is a top down view of an absorbent core 56 and core wrap 200, FIG. 10B is a cross-sectional view of the absorbent core 56 taken along line 10B-10B, and FIG. 100 is a cross-sectional view of the absorbent core 56 taken along line 100-100. As illustrated in FIGS. 10A, 10B, and 10C, the absorbent core 56 can be shaped so as to be non-symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is greater than the second maximum transverse direction width 116 of the absorbent core 56. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is less than 3 mm. The interior surfaces, 214 and 224, of the core wrap side edges, 210 and 220, respectively, can be spaced apart from the longitudinal direction side edges, 98 and 100, of the absorbent core at the second maximum transverse direction width 116 by a distance 212 and 222, respectively, which is at least 3.5 mm. In the exemplary embodiment illustrated in FIGS. 10A, 10B, and 100, distances 212 and 222 are greater than distances 216 and 226.

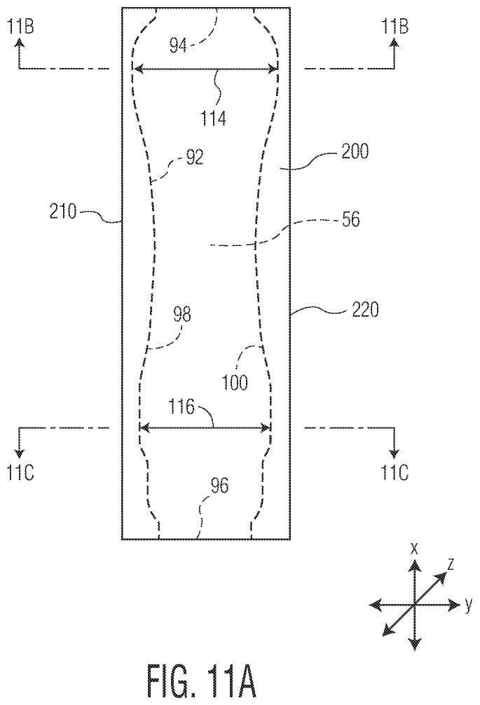

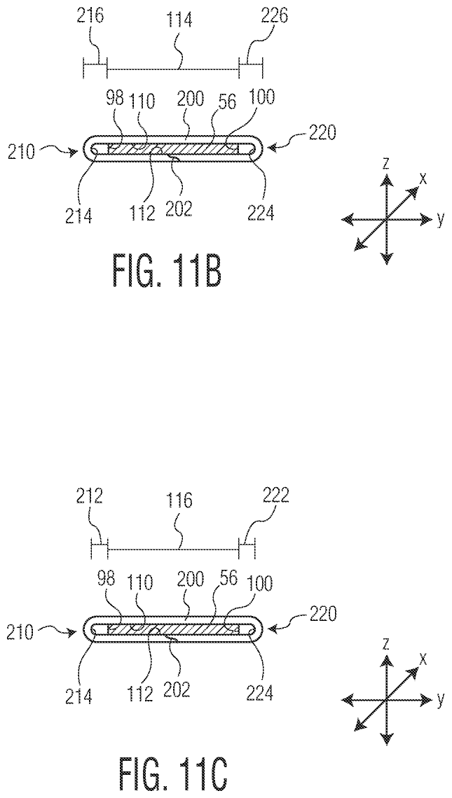

Referring to FIGS. 11A, 11B, and 11C, FIG. 11A is a top down view of an absorbent core 56 and core wrap 200, FIG. 11B is a cross-sectional view of the absorbent core 56 taken along line 11B-11B, and FIG. 11C is a cross-sectional view of the absorbent core 56 taken along line 11C-11C. As illustrated in FIGS. 11A, 11B, and 11C, the absorbent core 56 can be shaped so as to be non-symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is greater than the second maximum transverse direction width 116 of the absorbent core 56. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is at least 3.5 mm. The interior surfaces, 214 and 224, of the core wrap side edges, 210 and 220, respectively, can be spaced apart from the longitudinal direction side edges, 98 and 100, of the absorbent core at the second maximum transverse direction width 116 by a distance 212 and 222, respectively, which is at least 3.5 mm. In the exemplary embodiment illustrated in FIGS. 11A, 11B, and 11C, distances 212 and 222 are greater than distances 216 and 226.

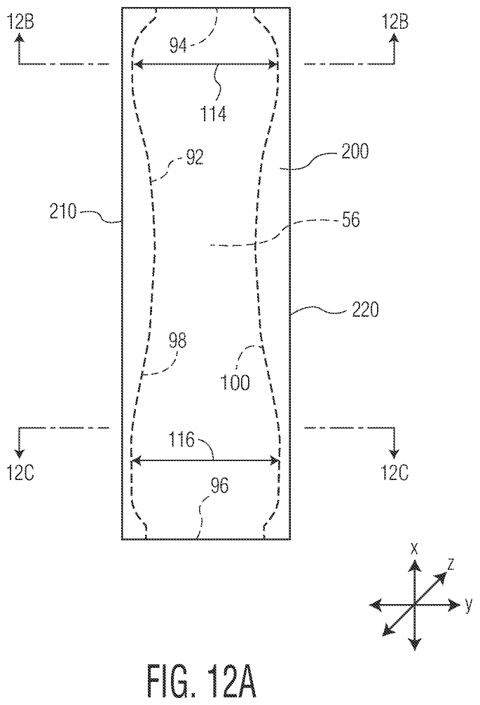

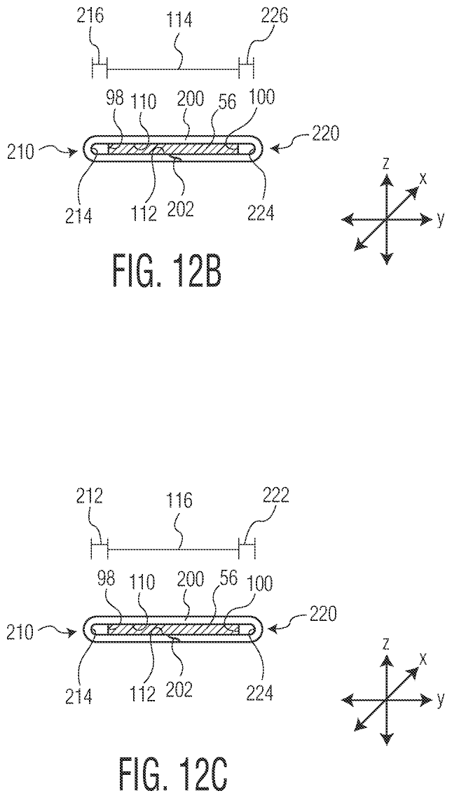

Referring to FIGS. 12A, 12B, and 12C, FIG. 12A is a top down view of an absorbent core 56 and core wrap 200, FIG. 12B is a cross-sectional view of the absorbent core 56 taken along line 12B-12B, and FIG. 12C is a cross-sectional view of the absorbent core 56 taken along line 12C-12C. As illustrated in FIGS. 12A, 12B, and 12C, the absorbent core 56 can be shaped so as to be non-symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is the same as the second maximum transverse direction width 116 of the absorbent core 56. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is at least 3.5 mm. The interior surfaces, 214 and 224, of the core wrap side edges, 210 and 220, respectively, can be spaced apart from the longitudinal direction side edges, 98 and 100, of the absorbent core at the second maximum transverse direction width 116 by a distance 212 and 222, respectively, which is at least 3.5 mm. In the exemplary embodiment illustrated in FIGS. 12A, 12B, and 12C, distances 212 and 222 are the same as distances 216 and 226.

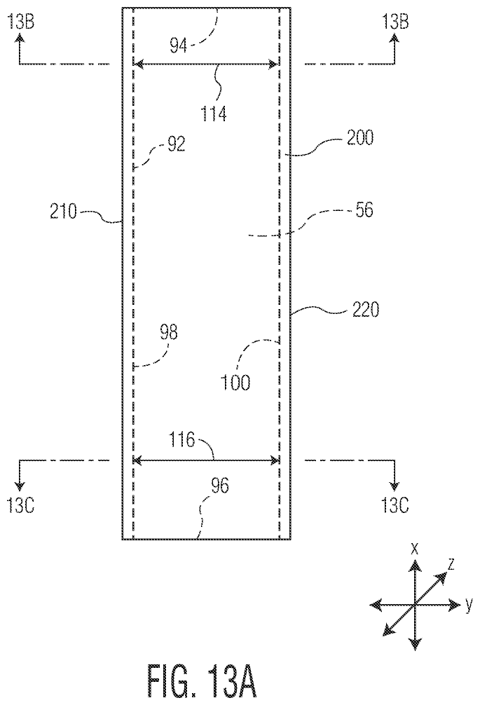

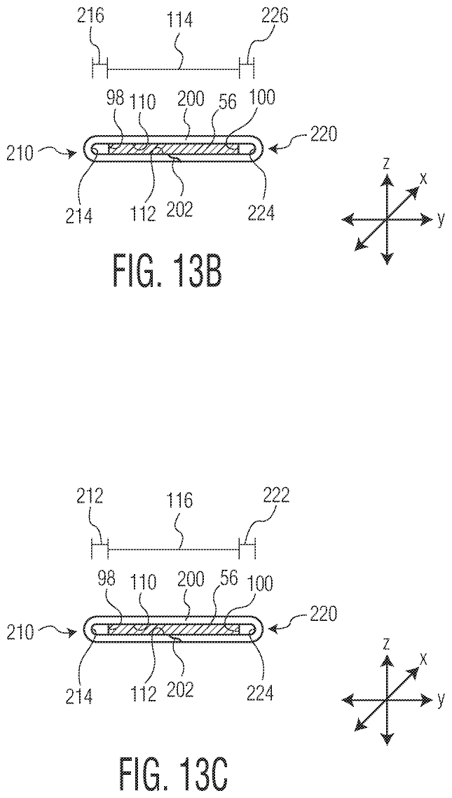

Referring to FIGS. 13A, 13B, and 13C, FIG. 13A is a top down view of an absorbent core 56 and core wrap 200, FIG. 13B is a cross-sectional view of the absorbent core 56 taken along line 13B-13B, and FIG. 13C is a cross-sectional view of the absorbent core 56 taken along line 13C-13C. As illustrated in FIGS. 13A, 13B, and 13C, the absorbent core 56 can be shaped so as to be symmetrical about its transverse axis and can have a first maximum transverse direction width 114 at one longitudinal direction end of the absorbent core 56 and can have a second maximum transverse direction width 116 at the opposite longitudinal direction end of the absorbent core 56. The first maximum transverse direction width 114 of the absorbent core 56 is the same as the second maximum transverse direction width 116 of the absorbent core 56. In the illustrated embodiment, the interior surfaces, 214 and 224, of the core wrap 200 side edges, 210 and 220, respectively, can be spaced apart from the longitudinal direction side edges, 98 and 100, of the absorbent core 56 at the first maximum transverse direction width 114 by a distance 216 and 226, respectively, which is at least 3.5 mm. The interior surfaces, 214 and 224, of the core wrap side edges, 210 and 220, respectively, can be spaced apart from the longitudinal direction side edges, 98 and 100, of the absorbent core at the first maximum transverse direction width 116 by a distance 212 and 222, respectively, which is at least 3.5 mm. In the exemplary embodiment illustrated in FIGS. 13A, 13B, and 13C, distances 212 and 222 are the same as distances 216 and 226.

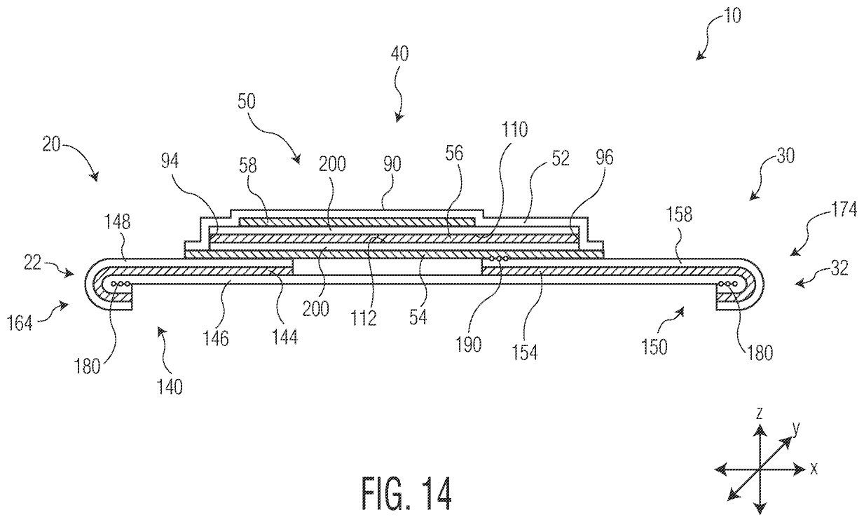

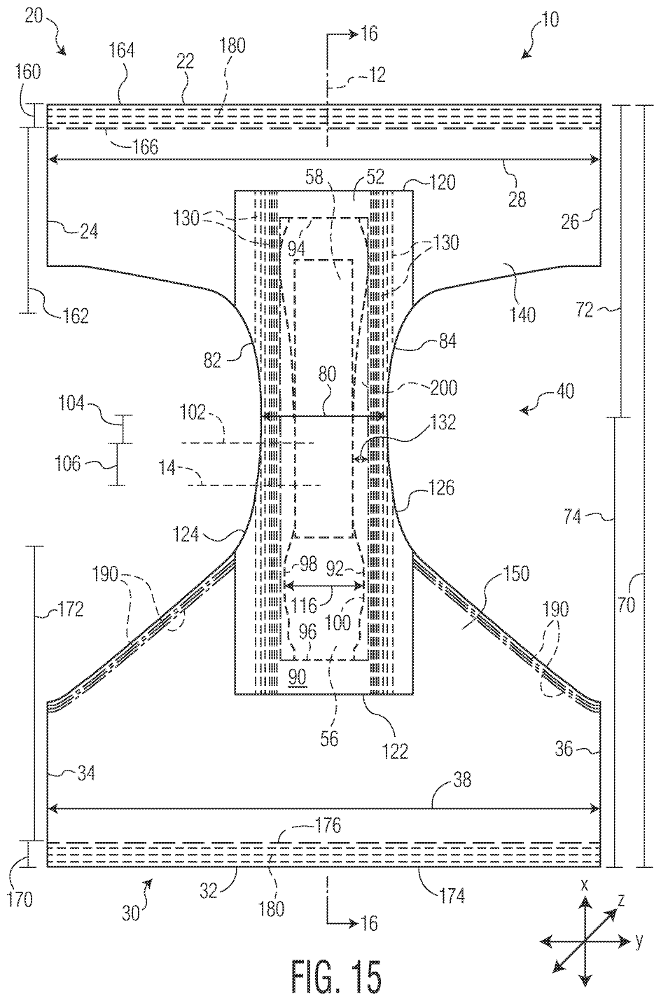

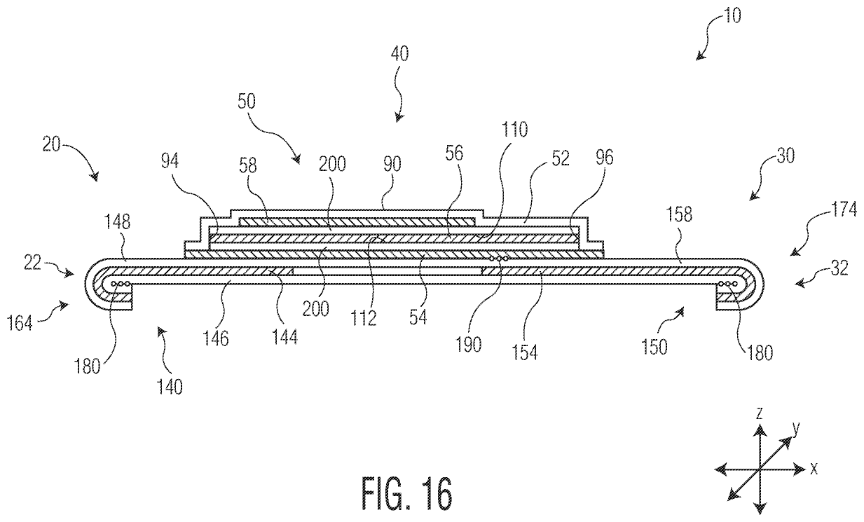

Referring to FIGS. 14-16, in various embodiments, the first waist region 20 and the second waist region 30 can be connected to each other via at least one of the materials forming the elastomeric panels, 130 and 140, respectively, of the first waist region 20 and the second waist region 30, respectively. FIG. 14 provides an exemplary illustration in which one of the nonwoven layers, such as nonwoven layer 146 is common to both elastomeric panels, 140 and 150, of each of the first waist region 20 and second waist region 30, respectively, and longitudinally extends from the first waist region 20, through the crotch region 40, and to the second waist region 30. FIGS. 15 and 16 provide an exemplary illustration of an absorbent article 10 in which both of the nonwoven layers, 146 and 148, are common to each of the elastomeric panels, 140 and 150, of the first waist region 20 and second waist region 30 and longitudinally extend from the first waist region 20, through the crotch region 40, and to the second waist region 30.

In various embodiments, the second waist region 30 can further have a leg elastic 190. In various embodiments, the second waist region 30 can have 1, 2, 3, 4, 5, or 6 leg elastics 190. Each leg elastic 190 can be a single strand, ribbon, or strip of elastomeric material. For example, the second waist region 30 illustrated in FIG. 3 illustrates three strands of leg elastics 190.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any documents is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

What is claimed is:

1. An absorbent article comprising:
a. a longitudinal direction and a transverse direction;
b. a longitudinal axis and a transverse axis;
c. a first waist region comprising a first waist edge, a first longitudinal direction side edge, a second longitudinal direction side edge transversely opposed to the first longitudinal direction side edge, a first elastomeric panel comprising a first elastic material positioned between a first nonwoven material and a second nonwoven material wherein the first elastic material is elastomeric in the transverse direction;
d. a second waist region comprising a second waist edge, a third longitudinal direction side edge, a fourth longitudinal direction side edge transversely opposed to the third longitudinal direction side edge, a second elastomeric panel comprising a second elastic material positioned between a third nonwoven material and a fourth nonwoven material, wherein the second elastic material is elastomeric in the transverse direction;
e. a first side seam formed by bonding the first longitudinal direction side edge of the first waist region to the third longitudinal direction side edge of the second waist region and a second side seam formed by bonding the second longitudinal direction side edge of the first waist region to the fourth longitudinal direction side edge of the second waist region;
f. a crotch region located between the first waist region and the second waist region and interconnecting the first waist region and the second waist region;
g. an article length measured from the first waist edge to the second waist edge; and
h. an absorbent assembly comprising:
i. an absorbent core comprising a first longitudinal direction side edge and a second longitudinal direction side edge transversely opposed to the first longitudinal direction side edge, a topsheet layer facing surface and a backsheet layer facing surface, wherein a first portion of the absorbent core is located in the crotch region, a second portion of the absorbent core is located in a portion of the first waist region and in an overlapping configuration with a portion of the first elastic material and comprises a first maximum transverse direction width, and a third portion of the absorbent core is located in a portion of the second waist region and in an overlapping configuration with a portion of the second elastic material and comprises a second maximum transverse direction width;
ii. a core wrap comprising a first side edge spaced apart from the first longitudinal direction side edge of the absorbent core by a distance of at least 3.5 mm in at least one of the first waist region or the second waist region when the absorbent article is in a transverse direction fully extended configuration, and a second side edge spaced apart from the second longitudinal direction side edge of the absorbent core by a distance of at least 3.5 mm in at least one of the first waist region or the second waist region when the absorbent article is in a transverse direction fully extended configuration; and wherein a portion of the core wrap is in a face-to-face relationship with the backsheet layer facing surface of the absorbent core.

2. The absorbent article of claim 1 wherein the first elastic material and the second elastic material is a plurality of elastomeric strands.

3. The absorbent article of claim 1 wherein the first elastic material and the second elastic material is a polymeric film sheet.

4. The absorbent article of claim 1 wherein the first maximum transverse direction width of the absorbent core is greater than the second maximum transverse direction width of the absorbent core.

5. The absorbent article of claim 1 wherein the first maximum transverse direction width of the absorbent core is the same as the second maximum transverse direction width of the absorbent core.

6. The absorbent article of claim 1 wherein the first maximum transverse direction width of the absorbent core is less than the second maximum transverse direction width of the absorbent core.

7. The absorbent article of claim 1 wherein, in the second waist region, the first side edge of the core wrap is spaced apart from the first longitudinal direction side edge of the absorbent core at the second maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the second maximum transverse direction width of the absorbent core by a distance of at least 3.5 mm when the absorbent article is in a transverse direction fully extended configuration.

8. The absorbent article of claim 7 wherein, in the first waist region, the first side edge of the core wrap is spaced apart from the first longitudinal direction side edge of the absorbent core at the first maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the first maximum transverse direction width of the absorbent core by a distance of at least 3.5 mm when the absorbent article is in a transverse direction fully extended configuration.

9. The absorbent article of claim 1 wherein, in each of the first waist region and the second waist region, the first side edge of the core wrap is spaced apart from the first longitudinal direction side edge of the absorbent core at each of the first maximum transverse direction width and the second maximum transverse direction width by a distance of at least 3.5 mm when the absorbent article is in a transverse direction fully extended configuration and the second side edge of the core wrap is spaced apart from the second longitudinal direction side edge of the absorbent core at each of the first maximum transverse direction width and the second maximum transverse direction width by a distance of at least 3.5 mm when the absorbent article is in a transverse direction fully extended configuration.

10. The absorbent article of claim 1 wherein, in the first waist region, the first side edge of the core wrap is spaced apart from the first longitudinal direction side edge of the absorbent core at the first maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the first maximum transverse direction width of the absorbent core by a distance of at least 3.5 mm when the absorbent article is in a transverse direction fully extended configuration.

11. The absorbent article of claim 10 wherein, in the second waist region, the first side edge of the core wrap is spaced apart from the first longitudinal direction side edge of the absorbent core at the second maximum transverse direction width of the absorbent core by a distance of at least 3.5 mm when the absorbent article is in a transverse direction fully extended configuration and the second side edge of the core wrap is spaced apart from the second longitudinal direction side edge of the absorbent core at the second maximum transverse direction width of the absorbent core by a distance of at least 3.5 mm when the absorbent article is in a transverse direction fully extended configuration.

12. The absorbent article of claim 1 further comprising an absorbent article narrowest width in the transverse direction positioned in the crotch region and between the transverse axis and the first waist edge.

13. The absorbent article of claim 12 further comprising a first waist region width in the transverse direction from the first longitudinal direction side edge to the second longitudinal direction side edge wherein the absorbent article narrowest width is less than 25% of the first waist region width.

14. The absorbent article of claim 12 further comprising a first article sub-length measured from the first waist edge to the absorbent article narrowest width and a second article sub-length measured from the second waist edge to the absorbent article narrowest width wherein the first article sub-length is less than the second article sub-length.

15. The absorbent article of claim 14 wherein the first article sub-length is less than 45% of the article length and the second article sub-length is greater than 55% of the article length.

16. The absorbent article of claim 12 wherein the absorbent core further comprises an absorbent core midpoint which is positioned between the absorbent article narrowest width and the transverse axis.

17. The absorbent article of claim 16 wherein the absorbent core midpoint is offset in the longitudinal direction from the absorbent article narrowest width by a distance from 1% to 5% of the article length.

18. The absorbent article of claim 16 wherein the absorbent core midpoint is offset in the longitudinal direction from the transverse axis of the absorbent article by a distance from 6% to 15% of the article length.
